(19) United States
(12) Patent Application Publication
(10) Pub. No.: US 2014/0371418 A1
(43) Pub. Date: Dec. 18, 2014

(54) BIOLOGICAL SYNTHESIS OF P-AMINOBENZOIC ACID, P-AMINOPHENOL, N-(4-HYDROXYPHENYL)ETHANAMIDE AND DERIVATIVES THEREOF

(71) Applicant: pAromatics, LLC, Dover, DE (US)

(72) Inventors: Wing On Ng, Mason, MI (US); Shingo Watanabe, Green Bay, WI (US); Robert W.R. Humphreys, Point Lookout, NY (US); Steven C. Slater, Middleton, WI (US)

(73) Assignee: pAromatics, LLC, Dover, DE (US)

(21) Appl. No.: 14/370,683

(22) PCT Filed: Jan. 4, 2013
(86) PCT No.: PCT/US2013/020389

§ 371 Date: Jul. 3, 2014

Related U.S. Application Data
(60) Provisional application No. 61/583,422, filed on Jan. 5, 2012.
(60) Provisional application No. 61/614,344, filed on Mar. 22, 2012.

(51) Int. Cl. C12P 7/40; C08G 18/32; C07C 263/00; C08G 18/73; C07C 209/46; C07C 209/68

(52) U.S. Cl. CPC C12P 7/40; C07C 209/46; C07C 209/68; C07C 263/00; C08G 18/73; C08G 18/3221; C08G 18/3206
USPC 528/84; 435/252.3; 435/252.33; 435/254.11; 435/254.21; 435/254.3; 435/136; 564/395; 564/305; 558/299; 528/85

(57) ABSTRACT

The invention generally relates to biological engineering of microorganisms and production of chemical compounds therefrom. More particularly, the invention relates to novel genetically engineered microorganisms for the fermentative production of p-aminobenzoic acid and related compounds from fermentable carbon substrates. The biologically derived PABA and related compounds from fermentable carbon substrates can be used in a number of applications including as a food supplement or raw materials for the syntheses of other industrial chemicals or polymers.

### PRIORITY CLAIMS AND RELATED PATENT APPLICATIONS

This application is the national phase of PCT/US13/20389, filed Jan. 4, 2013, which claims the benefit of priority from U.S. Provisional Application Ser. Nos. 61/583,422, filed on Jan. 5, 2012, and 61/614,344, filed on Mar. 22, 2012, the entire content of each of which is incorporated herein by reference in its entirety.

### TECHNICAL FIELDS OF THE INVENTION

The invention generally relates to biological engineering of microorganisms and production of chemical compounds therefrom. More particularly, the invention relates to novel genetically engineered microorganisms for the fermentative production of p-aminobenzoic acid (PABA, 4-aminobenzoic acid, Vitamin Bₓ), p-aminophenol, N-(4-hydroxyphenyl)ethanamide(acetaminophen or paracetamol) and related compounds from fermentable carbon substrates.

### BACKGROUND OF THE INVENTION

PABA is a C₇ aromatic compound, used commercially as a food supplement as well as precursors for the synthesis of azo dyes, folic acid and other industrial chemicals. Industrial production of PABA is mainly derived from 4-nitrobenzoic acid or terephthalic acid, both of which are derivatives of petroleum products. (Maki, T., K. Takeda (2000). Benzoic Acid And Derivatives. Ullmann's Encyclopedia Of Industrial Chemistry, Wiley-VCH Verlag GmbH & Co.) Currently, there is no renewable or biologically derived source of PABA available commercially.

PABA is a natural metabolite in the shikimic acid pathway and an essential precursor for the biosynthesis for the vitamin folic acid. (Green, et al. 1992 “Characterization And Sequence Of Escherichia Coli pabC, The Gene Encoding Aminodeoxychorismate Lyase, A Pyridoxal Phosphate-Containing Enzyme” Journal Of Bacteriology 174(16): 5317-5323.) The biosynthetic pathway of PABA is relatively well understood in both prokaryotes and eukaryotes (for example in the yeast Saccharomyces cerevisiae). (See, for example in Escherichia coli, Ye, et al. 1990 “P-Aminobenzoate Synthesis In Escherichia Coli: Purification And Characterization Of pabB As Aminodeoxychorismate Synthase And Enzyme X As Aminodeoxychorismate Iyase” Proceedings Of The National Academy Of Sciences Of The United States Of America 87(23): 9391-9395.)

Current production methods of PABA, aniline, and PPD rely on chemical synthesis from petroleum-derived chemicals. Multiple chemical steps involved in the chemical synthesis result in high production cost of the chemicals. In addition, non-specific chemical substitution on the aromatic ring results in the production of side products thereby reducing the yield. Hazardous chemical intermediates, solvents and wastes associated with the conventional chemical synthesis pose substantial impacts on the environment. Reliance on petroleum-derived raw materials suffers from unpredictable cost fluctuations as a result of the long-term uncertainty in global petroleum price.

Biologically-derived PABA made from fermentable carbon substrates, in contrast, has the potential to cost less to produce. Highly-specific biochemical conversions help to minimize the production of side products. Also, the use of hazardous chemicals and the resulting waste are kept to a minimum. Besides the above advantages, the bio-based process poses much less overall impact to the environment. Biologically derived PABA can serve as a versatile substrate for other chemical synthesis. It can be converted into high-valued polymer without further chemical modification. (Kwoleck 1974 “Wholly aromatic carbocyclic polycarbonate fiber having orientation angle of less than about 45 degrees”, U.S. Pat. No. 3,819,587.) PPD is one of the monomers used for the synthesis of Aramid polymers.

There is no known example of the large-scale biological production of PABA. As a precursor for the synthesis of folic acid, PABA was used as a supplement for folic acid production in microorganisms. (Miyata, et al. 1999 “Method for producing folic acid” U.S. Pat. No. 5,968,788.) In another example, folic acid production was increased by the overexpression of PABA biosynthetic genes, pabA and pabBC. (Wegkamp, et al. 2007 “Characterization Of The Role Of Para-Aminobenzoic Acid Biosynthesis In Folate Production By Lactococcus Lactis” Applied And Environmental Microbiology 73(8): 2673-2681.) Sulfonamide-resistant bacteria were known to produce an elevated level of PABA to overcome the inhibition of sulfonamide on folic acid synthesis (Leskowitz, et al. 1952 “The Isolation And Identification Of Para-Aminobenzoic Acid Produced By Staphylococci Resistant To Sulfonamide” Journal Of Experimental Medicine 95(3): 247-250).

PPD is used for a variety of applications, such as cosmetics, antioxidants, fuel additives and dye stuff and a raw material for specialty high-performance thermoplastics such as the aramids. Commonly PPD is produced from benzene via chlorobenzene and para-nitrochlorobenzene followed by nitration, amination and hydrogenation. Nitrochlorobenzene is produced from chlorobenzene with ortho-, meta- and para-isomers at the best reported ratio of 38:1:61. (Demuth, et al. 2003 “Continuous adiabatic process for preparing nitrochlorobenzene” U.S. Pat. No. 6,586,645.) This route produce significant amount of by-products, such as ortho and meta. In addition to productivity of para-nitrochlorobenzene, unfavorable halogenated compound is produced. The synthesis route from 4-nitrochlorobenzene to PPD is shown below (i).

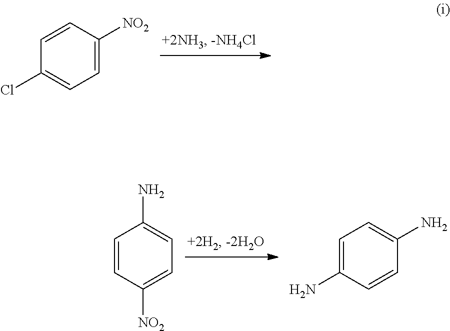 [SMILES: O=[N+]([O-])c1ccc(Cl)cc1 ; Nc1ccc([N+](=O)[O-])cc1 ; Nc1ccc(N)cc1]

The other synthesis method is reaction of benzamide and nitrobenzene in presence of a base. (Stern, et al. 1993 “Amination Of Nitrobenzene Via Nucleophilic Aromatic-Substitution For Hydrogen—Direct Formation Of Aromatic Amide Bonds” Journal of Organic Chemistry 58(24): 6883-6888; Stern, M. K. 1994 “Nucleophilic Aromatic-Substitution For Hydrogen—New Halide-Free Routes For Production of Aromatic-Amines. Benign By Design: Alternative Synthetic Design For Pollution Prevention.” P. T. Anastas And C. A. Farris. 577: 133-143.) Advantages of this method are its relatively higher selectivity to produce PPD and it does not require halogen usage. However, the process comprises multiple steps starting with two molecules, and the tetramethylammonium base is relatively costly.

An alternative synthesis route is amination of hydroquinone. A selectivity of 97% was reported in liquid phase, and 98% of phenol to aniline and 98% of aniline to PPD selectivity in vapor phase was also reported. (Weil 1983 “Process for 1,4-phenylenediamine” U.S. Pat. No. 4,400,537; Hidaka, et al. 2001 “Method For Producing Aromatic Amino Compound” JP Patent No. 2001151735.) Although both liquid and gas phase syntheses are highly selective, they are not productive due to significantly low concentration of raw materials.

### SUMMARY OF THE INVENTION

The invention provides novel genetically engineered microorganisms for fermentative production of aromatic molecules from biomass-based sugars. For example, the invention provides genetically engineered strains of yeast as biocatalysts that are suitable for efficient fermentative production of p-aminobenzoic acid (PABA, 4-aminobenzoic acid, Vitamin Bₓ), p-aminophenol, N-(4-hydroxyphenyl)ethanamide(acetaminophen or paracetamol) and other compounds from fermentable carbon substrates. The biologically derived PABA can be used in a number of applications including as a food supplement or raw materials for the syntheses of other industrial chemicals or polymers. Furthermore, the present invention relates to preparation methods of aromatic diamines, in particular para-phenylenediamine(p-phenylenediamine or PPD), by decarboxylation and amination of aminobenzoic acid in the presence of a precious metal and base metal catalyst. In particular, PABA can be chemically converted to PPD, and the chemical processes for the synthesis of the polymer and PPD from PABA is equally applicable to petroleum-derived PABA. p-Aminophenol can also be aminated chemically to PPD, providing an additional route for renewable PPD.

In addition to PPD, biologically-derived PABA can also serve as precursors to the synthesis of other chemicals, for example, methylenedianiline (MDA) and methylene diphenyl diisocyanate (MDI).

In one aspect, the invention generally relates to a recombinant microbial host cell capable of converting a fermentable carbon substrate to p-aminobenzoic acid biologically. The recombinant microbial host cell may be any suitable host cell, for example, a bacterium, a cyanobacterium, an archaeon, or a fungus.

In another aspect, the invention generally relates to a method for fermentative production of p-aminobenzoic acid comprising converting a fermentable carbon substrate to p-aminobenzoic acid by biological fermentation using a recombinant microbial host cell.

In yet another aspect, the invention generally relates to a method for making p-phenylenediamines comprising reacting ammonia and biologically-derived p-aminobenzoic acid in the presence of a precious metal catalyst on a support.

In yet another embodiment, the invention generally relates to a method for making p-phenylenediamines comprising reacting ammonia and petroleum-derived p-aminobenzoic acid in the presence of a precious metal catalyst on a support.

In yet another embodiment, the invention generally relates to a method for making p-phenylenediamines comprising reacting biologically-derived p-aminophenol (PAP) of claim 35 and ammonia in the presence of a precious metal catalyst on a support.

In yet another aspect, the invention generally relates to a method for making aniline comprising decarboxylating p-aminobenzoic acid.

In yet another aspect, the invention generally relates to a method for preparing p-phenylenediamine comprising amination of N-(4-hydroxyphenyl)ethanamide. In certain embodiments, the amination of N-(4-hydroxyphenyl)ethanamide is carried out in the presence of a precious metal catalyst on a support.

### BRIEF DESCRIPTION OF THE DRAWINGS

FIG. 1 shows a schematic depiction of exemplary renewable chemicals that can be derived from bio-based (bio-) PABA, bio-p-aminophenol, and bio-acetaminophen.

FIG. 2 shows a schematic depiction of the shikimic acid pathway in E. coli.

FIG. 3 shows a schematic depiction of a modified shikimic acid pathway for the production of PABA in E. coli.

FIG. 4 shows a schematic depiction of the shikimic acid pathway in S. cerevisiae.

FIG. 5 shows a schematic depiction of a modified shikimic acid pathway for the production of PABA in S. cerevisiae.

### DETAILED DESCRIPTION OF THE INVENTION

The invention is based, in part, on novel genetically engineered microorganisms for fermentative production of aromatic molecules from biomass-based materials. The invention provides efficient biocatalysts for the production of PABA and related compounds, which can serve as a versatile and renewable feedstock for production of a wide range of valuable, commercial aromatic amine-based chemicals, monomers, polymers and dye, pesticide and pharmaceutical intermediates through additional biological and chemical conversions. These bio-based chemicals are cost competitive, drop-in replacements for the current, petroleum derived counterparts.

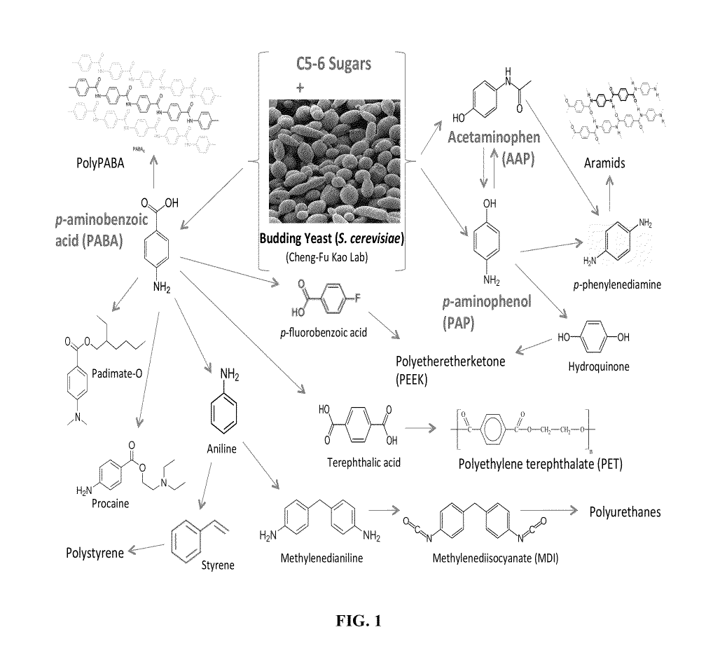

FIG. 1 shows exemplary renewable chemicals that can be derived from bio-based (bio-) PABA, bio-p-aminophenol, and bio-acetaminophen. A well-characterized hydroxylase from the common button mushroom (Agaricus bisporus) can perform a controlled oxidative decarboxylation on PABA to produce para-aminophenol (PAP), an oxidative transformation that is difficult to implement with conventional chemistry due to the sensitivity of the amine function on PABA. (Tsuji, et al. 1985 Biochem. & Biophys. Res. Comm. 130(2): p. 633-639.) The resulting PAP can be acetylated with an arylamine N-acetyltransferase, to form acetaminophen (AAP), which can be converted into PAP and substituted for PAP in chemical conversion of PAP to many derivatives. (Mulyono, et al., 2007 J Biosci. Bioeng. 103(2): p. 147-54.)

PAP contains both amino and hydroxyl groups and can be converted into p-phenylenediamine (PPD) by reaction with ammonia in the presence of a noble metal catalyst. (Mitsutatsu, et al., 1988, Production of p-phenylenediamines, J.P. Office, Editor, Mitsui Petrochemical Co. Ltd.: Japan.) Bio-PPD could be key component of lower cost, 100% renewable para-aramid, a very important engineering polymer used in ultra-high strength fiber applications. Replacement of the amino group of PAP to give hydroquinone (HQ) can be accomplished smoothly by heating PAP and an organic sulfonic acid at elevated temperature in water. (Biller, 1981, Hydroquinone by hydrolysis of p-aminophenol or salts U.P.a.T. Office, Editor, United States.) Hydroquinone is the second monomer component of the engineering polymer PEEK (see below for first component) and is a critical component in industrial antioxidant technology. PAP can also be converted easily to p-fluorophenol, an important pharmaceutical, pesticide and dye intermediate, via the diazonium salt. (Langlois, 2000, “Introduction of Fluorine via Diazonium Compounds (Fluorodediazoniation)”, in Science of Synthesis: Houben-Weyl Methods of Molecular Transformations, E. J. Thomas, Editor, Thieme Publishing Group. p. 686-740.)

PABA contains carboxylate and amine functions, both of which can be eliminated, providing access to aniline and benzoic acid families of aromatic chemicals and monomers. For example, PABA decarboxylates to aniline by heating in acidic aqueous solution. (Zhao, et al., 2001 Molecules 6(12): p. M246; Schiemann, et al. 1943 Organic Synthesis 2: p. 299-301.) Aniline is a key raw material for a wide range of commercial chemicals and monomers, including: 4,4-methylenedianiline (MDA), used in aromatic polyurethane foams, elastomers, and adhesives; aniline dyes and pigments; antioxidants, and herbicides. Since bio-sources for polyols and polyester polyols are available, bio-aniline offers an opportunity for 100% renewable versions of commercial polyurethane polymers.

PABA can be converted to the corresponding diazonium compound in high yield under mild, commercially practical conditions using low cost reagents. (Los, et al. 1967 Recueil des Travaux Chimiques des Pays-Bas 86(6): p. 622-640.) Reductive elimination of the diazo group gives benzoic acid directly. (Smith, M. B. and J. Mach, March's Advanced Organic Chemistry. 6 ed 2007, New York: Wiley Interscience.) The diazonium salt can be converted to many commercial benzoic acid derivatives, including:
4-fluorobenzoic acid, one of two monomers in the engineering polymer PEEK. The other monomer, hydroquinone, can be prepared from PAP, providing 100% renewable PEEK. (Schiemann, G. and W. Winkelmuller, p-Fluorobenzoic acid. Organic Synthesis, 1943. 2: p. 299-301.)
4-hydroxybenzoic acid and p-anisic acid derivatives. Applications include foods, fragrances and personal care.
Terephthalic acid (PTA), via catalytic carbonylation. Bio-PTA combined with bio-sourced ethylene glycol will afford 100% renewable PET. (Willi, A. V., Homogeneous Catalysis of Organic Reactions (mainly acid-base), in Comprehensive Chemical Kinetics, C. H. Bamford and C. F. H. Tipper, Editors. 1977, Elsevier. p. 72-82.)
Styrene derivatives, such as 4-carboxystyrene, via catalytic addition of ethylene. A similar reaction sequence can be applied to aniline to generate styrene. (id.)

The diazonium salt prepared from PABA as described herein can also be converted into many other p-substituted benzoic acid derivatives by reaction with appropriate reagents known to react with diazonium salts. Example of such derivatives include, but are not limited to, p-chlorobenzoic acid, p-bromobenzoic acid, p-hydroxybenzoic acid, p-mercaptobenzoic acid, p,p′dicarboxydiphenylsulfide, p-thiocyanatobenzoic acid, p,p′-dicarboxyazobenzene, and p-cyanobenzoic acid.

PABA can also be converted into polyPABA, a polyamide that has been commercialized for high performance fiber applications. (Pramanik, et al. 2004 Resonance 9(6): p. 39-50; Kwolek, S. L., POLY(p-BENZAMIDE) COMPOSITION, PROCESS AND PRODUCT, U.S. Pat. No. 3,600,350, 17 Aug. 1971; Pikl, et al., POLYMERIZATIONS AND POLYMERIZATION CONDITIONS, U.S. Pat. No. 3,541,056, 17 Nov. 1970; Morgan, P. W., POLY (1,4-BENZAMIDE) COPOLYMERS, U.S. Pat. No. 3,99,016, 9 Nov. 1976; Morgan, P. W., Process for preparing film- and fiber-forming poly(1,4-benzamide), U.S. Pat. No. 4,025,494, 24 May, 1977.)

Thus, the methods of the invention enable cost-effective production of aromatic amine-based chemicals, monomers, and polymers directly from biomass via efficient fermentation processes in high volume production. In addition to economic benefits, this disclosed technology eliminates many of the environmental, health, and safety drawbacks associated with conventional manufacturing routes through BTX (benzene/toluene/xylene), such as the volatility and toxicity associated with these aromatic hydrocarbons and the need for subsequent amination processes that must be employed to introduce the amine functionality.

For example, the biologically derived PABA can be used as a food supplement or raw materials for the syntheses of other industrial chemicals (e.g., azo dyes, procaine, acetaminophen). This biologically derived PABA can also be polymerized to form high-strength polymer. PABA can also be enzymatically converted further into p-aminophenol, which can serve as a precursor for other chemicals. Furthermore, the present invention relates to a preparation method of aromatic diamines, in particular PPD, by amination of p-aminophenol in the presence of a precious metal and base metal catalyst. In particular, p-aminophenol can be chemically converted to PPD, a monomer for the production of aramids. As disclosed herein, the chemical processes for the synthesis of the polymer and PPD from p-aminophenol are equally applicable to petroleum-derived p-aminophenol.

The present invention also relates to the preparation of aniline and aniline-based chemicals from biologically-derived or petroleum-derived PABA. For example, PABA can be decarboxylated to aniline in the presence of suitable catalysts. Suitable catalysts include acid catalysts such as hydrochloric, phosphoric, and sulfuric acids, organic acids such as p-toluenesulfonic acid, polymeric acid catalysts such as sulfonated polystyrene resins, and heterogeneous acidic catalysts such as silicas, zeolites, aluminas such as γ-alumina. The decarboxylation can be carried out in a variety of ways, such as in aqueous solution, in organic solvents, or in the melt.

The PABA-derived aniline can be converted to a broad range of aniline-based chemicals. An important example of such aniline-based chemicals is methylenedianiline from the condensation of aniline with formaldehyde in the presence of suitable catalysts. The aniline-formaldehyde condensation products can also include higher molecular weight condensation products incorporating more than two aniline molecules and more than one formaldehyde molecule as well as mixtures of different molecular weight aniline-formaldehyde condensation products. Such aniline condensation products are technologically important intermediates for production of isocyanates that are critical to production of polyurethanes. For example, methylenedianiline can be converted into methylene diphenyl diisocyanate, a critical component in many high performance polyurethanes, using phosgene in an appropriate solvent. These aniline-formaldehyde condensation products and the corresponding isocyanates can be prepared from aniline derived from biologically-derived PABA, biologically-derived formaldehyde and biologically-derived phosgene, thus providing 100% biologically-sourced, and hence 100% renewable, aniline-formaldehyde condensation products and the corresponding isocyanates. The biologically-derived formaldehyde can be made from fermentation-derived methanol using dehydrogenation catalysts while the biologically-derived phosgene can be obtained from biologically-sourced carbon monoxide (from CO₂ using the water-gas shift reaction) and chlorine.

The present invention also relates to a method for producing aniline and aniline derivatives such as aniline-formaldehyde condensation products directly from PABA, including PABA derived from biological and petroleum sources. Reaction of biologically-derived PABA and bio-derived formaldehyde followed by biologically-derived phosgene will produce 100% biologically-derived aniline-formaldehyde condensation products and isocyanates, respectively. Finally, if biologically-sourced diols and polyols are used in preparation of polyurethanes from the PABA-derived isocyanates disclosed herein, then this invention allows the preparation of 100% biologically-sourced, and hence 100% renewable, polyurethanes. Such diols and polyols are well known in the art and include, for example, 1,3-propanediol, 1,4-butanediol, 1,6-hexanediol, fatty acid dimer and trimer diols and polyols, and polyester diols and polyols derived from biologically-sourced diols and diacids. These and other such diols and polyols, whether petroleum or biologically sourced, are incorporated into this invention to prepare partially or 100% biologically-derived, and hence partially or 100% renewable, polyurethanes when reacted with the PABA-derived isocyanates described herein.

Thus, in one aspect, the invention generally relates to a recombinant microbial host cell capable of converting a fermentable carbon substrate to p-aminobenzoic acid biologically.

The recombinant microbial host cell may be any suitable host cell, for example, a bacterium, a cyanobacterium, an archaeon, or a fungus. In certain embodiments, the microbial host cell is a Gram-positive bacterium. In certain embodiments, the microbial host cell is Escherichia coli. In certain embodiments, the E. coli host cell has been subjected to directed evolution and is characterized by an enhanced production of, and/or tolerance to, p-aminobenzoic acid.

In certain embodiments, the microbial host cell is Saccharomyces cerevisiae. In certain embodiments, the S. cerevisiae host cell has been subjected to directed evolution and is characterized by an enhanced production of, and/or tolerance to, p-aminobenzoic acid.

In certain embodiments, the microbial host cell is a filamentous fungus.

In certain embodiments, the microbial host cell is Kluyveromyces lactis. In certain embodiments, the microbial host cell is Aspergillus niger. In certain embodiments, the microbial host cell is Synechocystis sp. (e.g., Strain PCC 6803).

In another aspect, the invention generally relates to a method for fermentative production of p-aminobenzoic acid comprising converting a fermentable carbon substrate to p-aminobenzoic acid by biological fermentation using a recombinant microbial host cell.

In certain embodiments, the recombinant microbial host cell is E. coli, wherein the recombinant E. coli host cell is characterized by an inactivated 7,8-dihyropteroate synthase by mutation or enzymatic inhibition thereby preventing conversion of p-aminobenzoic acid to 7,8-dihyropteroate. In certain embodiments, the recombinant E. coli host cell is a 7,8-dihyropteroate synthase mutant requiring supplementation of methionine, glycine, thymidine, and pantothenate to maintain cell viability. In certain embodiments, the 7,8-dihyropteroate synthase mutant is rescued with folic acid transporters from Arabidopsis thaliana or Synechocystis sp. PCC6803 in the presence of (6R,6S)-5-formyl-tetrahydrofolic acid or folic acid.

In certain embodiments, the 7,8-dihyropteroate synthase mutant is characterized by increased activities of the aminodeoxychorismate synthase (pabA and pabB) and 4-amino-4-deoxychorismate lyase (pabC) by overexpression of corresponding genes that enhance conversion of chorismic acid to p-aminobenzoic acid. In certain embodiments, gene fusions between pabA and pabB (pabAB) as found in actinomyces, Plasmodium falciparum, and Arabidopsis thaliana enhance conversion of chorismic acid to p-aminobenzoic acid.

In certain embodiments, the recombinant E. coli host cell is characterized by a mutated anthranilate synthase with altered enzymatic activity that catalyses production of p-aminobenzoic acid is used in place of the aminodeoxychorismate synthase and 4-amino-4-deoxychorismate lyase activities.

In certain embodiments, the recombinant microbial host cell is S. cerevisiae.—In certain embodiments, the recombinant S. cerevisiae host cell is characterized by an inactivated the 7,8-dihyropteroate synthase activity by mutation or enzymatic inhibitors to prevent further conversion of p-aminobenzoic acid to 7,8-dihyropteroate. In certain embodiments, the recombinant S. cerevisiae host cell is a 7,8-dihyropteroate synthase mutant requiring supplementation of (6R,6S)-5-formyl-tetrahydrofolic acid or folic acid.

In certain embodiments, the 7,8-dihyropteroate synthase mutant is characterized by increased activities of aminodeoxychorismate synthase and 4-amino-4-deoxychorismate lyase activities by overexpression of corresponding genes that enhance conversion of chorismic acid to p-aminobenzoic acid. In certain embodiments, the 7,8-dihyropteroate synthase mutant is characterized by a mutated anthranilate synthase that catalyses production of p-aminobenzoic acid in place of aminodeoxychorismate synthase and 4-amino-4-deoxychorismate lyase activities.

In certain embodiments, the fermentable carbon substrate is selected from the group consisting of monosaccharides, oligosaccharides and polysaccharides.—In certain embodiments, the fermentable carbon substrate comprises a sugar derived from biomass. In certain embodiments, the fermentable carbon substrate comprise glucose, fructose or sucrose.

The fermentation can be carried out under dissolved oxygen concentration between 0-100% saturation (e.g., about 1%, 5%, 10%, 20%, 30%, 40%, 50%, 60%, 70%, 80%, 90%, 95%, 100%). The fermentation can be carried out in minimal medium supplemented with all necessary nutrients and maintained at a pH between about 1 to about 10 (e.g., about 1, 2, 3, 4, 5, 6, 7, 8, 9, 10).

In certain embodiments, p-aminobenzoic acid produced in the fermentation is purified by one or a combination of: precipitation at the isoelectric point of PABA, ion-exchange chromatography, and crystallization. In certain embodiments, p-aminobenzoic acid produced in the fermentation comprises up to 1 part per trillion of ¹⁴C.

In certain embodiments, the method further comprises purifying p-aminobenzoic acid produced in the fermentation and polymerizing the purified p-aminobenzoic acid to form a homopolymer or a heteropolymer.

In certain embodiments, the method further comprises purifying p-aminobenzoic acid produced in the fermentation and reacting the purified p-aminobenzoic acid with 2-diethylaminoethanol in the presence of sodium ethoxide to form procaine.

In certain embodiments, the method further comprises purifying p-aminobenzoic acid produced in the fermentation and chemically transforming the purified p-aminobenzoic acid to make folic acid, an azo dye or Padimate O.

In certain embodiments, the method further comprises converting p-aminobenzoic acid produced in the recombinant host organism to p-aminophenol by 4-aminobenzoate 1-monooxygenase (EC 1.14.13.27). In certain embodiments, the 4-aminobenzoate 1-monooxygenase is from Agaricus bisporus.

In certain embodiments, the method further comprises converting p-aminophenol to N-(4-hydroxyphenyl)ethanamide by arylamine N-acetyltransferases (EC 2.3.1.5). In certain embodiments, the arylamine N-acetyltransferases is NAT-a and NAT-b from Bacillus cereus Strain 10-L-2.

In certain embodiments, the recombinant microbial host cell is characterized by a S. cerevisiae vector expressing a DAHP synthase isozyme aroF^{FBR} from E. coli that is insensitive to feedback inhibition by tyrosine and aromatic amino acids.

In yet another aspect, the invention generally relates to a method for making p-phenylenediamines comprising reacting ammonia and biologically-derived p-aminobenzoic acid in the presence of a precious metal catalyst on a support.

In yet another embodiment, the invention generally relates to a method for making p-phenylenediamines comprising reacting ammonia and petroleum-derived p-aminobenzoic acid in the presence of a precious metal catalyst on a support.

In yet another embodiment, the invention generally relates to a method for making p-phenylenediamines comprising reacting biologically-derived p-aminophenol (PAP) of Claim 35 and ammonia in the presence of a precious metal catalyst on a support.

The precious metal catalyst may be any suitable metal catalyst, for example, Ru, Pd, Pt, Rh, Re, Au, Ir, Ni, Cu, Cr, and Co.

The support may be any suitable material, for example, activated carbon, SiO₂, Al₂O₃, TiO₂, ZrO₂, Nb₂O₅, Y₂O₃, and CeO₂.

The catalyst may be used in any suitable amount, for example, from about 0.01 wt % to about 20 wt % (e.g., about 0.5 wt %, 1 wt %, 5 wt %, 10 wt %, 15 wt %, 20 wt %) of p-aminobenzoic acid.

In certain embodiments, the reaction temperature is in the range from ambient temperature to about 400° C. (e.g., about 25° C., 50° C., 100° C., 150° C., 200° C., 250° C., 300° C., 350° C., 400° C.).

In certain embodiments, the ammonia is present during reaction with pressure in the range from about 15 psi to about 100 psi (e.g., about 15 psi, 20 psi, 30 psi, 40 psi, 50 psi, 60 psi, 70 psi, 80 psi, 90 psi, 100 psi). In certain embodiments, the ammonia is produced from hydrogen and nitrogen and p-aminobenzoic acid is pre-decarboxylated prior to reaction with ammonia. In certain embodiments, hydrogen pressure is maintained in the range from about 15 psi to about 5000 psi (e.g., about 15 psi, 50 psi, 100 psi, 500 psi, 1000 psi, 2000 psi, 3000 psi, 4000 psi, 5000 psi). In certain embodiments, the reaction is performed in aqueous medium or in an organic solvent. In certain embodiments, the reaction mixture comprises a base (e.g., KOH, LiOH, or NaOH).

In yet another aspect, the invention generally relates to a method for making aniline comprising decarboxylating p-aminobenzoic acid.

In certain embodiments, the p-aminobenzoic acid is prepared from fermentation using a recombinant microbial host cell capable of converting a fermentable carbon substrate to p-aminobenzoic acid biologically. In certain embodiments, the decarboxylation is carried out thermally by heating in a solution or neat in a melt. In certain embodiments, the decarboxylation is carried out thermally in the presence of an acid catalyst.

In certain embodiments, the solution is made by dissolving p-aminobenzoic acid in water. In certain embodiments, the solution is made by dissolving p-aminobenzoic acid in a thermally stable organic solvent.

In certain embodiments, the acid catalyst is a hydrochloric acid, a sulfuric acid, or a phosphoric acid, or a mixture thereof. In certain embodiments, the acid catalyst is a polymeric catalyst. In certain embodiments, the acid catalyst is a sulfonated polystyrene. In certain embodiments, the acid catalyst is a heterogeneous catalyst. In certain embodiments, the heterogeneous catalyst is acidic silicas, zeolites, clays, γ-alumina, or a mixture thereof.

In certain embodiments, the aniline is isolated and purified by removing a solvent, if present, followed by distilling the aniline under vacuum. In certain embodiments, the aniline is isolated and purified by steam distillation. In certain embodiments, the water is substantially removed by distillation and the aniline is dissolved in an organic solvent, dried, and distilled under vacuum after the solvent is removed. In certain embodiments, the method further comprises treating aniline with formaldehyde in water in the presence of a catalyst to produce methylenedianiline and/or poly-methylenedianiline. In certain embodiments, the formaldehyde is produced from an organic carbon source. In certain embodiments, the formaldehyde is produced by catalytic dehydration of fermentation-derived methanol.

In certain embodiments, the catalyst is an acid catalyst for example, a Bronstead acid (e.g., a hydrochloric acid, a sulfuric acid, a phosphoric acid, or a polymeric resin). In certain embodiments, the polymeric resin is sulfonated polystyrene.

In certain embodiments, the method further comprises purifying methylenedianiline by fractional, vacuum distillation.

In certain embodiments, the method further comprises controlling the relative amounts of 4,4′-, 2,4′- and aniline-formaldehyde condensation products having more than two aniline molecules and more than one formaldehyde molecule incorporates.

In certain embodiments, the method further comprises converting methylenedianiline and poly-methylenedianiline to the corresponding isocyanates, including methylene diphenyl diisocyanate and poly-methylene diphenyl diisocyanate.

In certain embodiments, the methylene diphenyl diisocyanate and poly-methylene diphenyl diisocyanate are prepared from biologically-derived methylenedianiline and biologically-derived poly-methylenedianiline.

In certain embodiments, the method further comprises reacting methylenedianiline or poly-methylenedianiline with phosgene in an inert solvent to produce methylene diphenyl diisocyanate and poly-methylene diphenyl diisocyanate.

In certain embodiments, the phosgene is prepared from a source of organic carbon. In certain embodiments, the phosgene is prepared from biologically-sourced carbon monoxide and chlorine, where the carbon monoxide is prepared from carbon dioxide via the water-gas shift reaction.

In certain embodiments, the inert solvent comprises one or more of benzene, toluene, xylenes, chlorobenzene, and dichlorobenzene.

In certain embodiments, poly-methylenedianiline is rich in the 2,4′-isomer.

In certain embodiments, the method further comprises distilling methylene diphenyl diisocyanate. In certain embodiments, the method further comprises fractionally distilling methylene diphenyl diisocyanate.

In certain embodiments, the method further comprises reacting methylene diphenyl diisocyanate or poly-methylene diphenyl diisocyanate with polyols or polyesterdiols to produce polyurethane polymers and prepolymers. In certain embodiments, the methylene diphenyl diisocyanate and poly-methylene diphenyl diisocyanate are partially or totally biologically-derived and the polyols and polyesterdiols are prepared from biologically sourced ethylene glycol, propanediol, butanediol, hexanediol, adipic acid, succinic acid, dimer and trimer acids, terephthalic acid, phthalic acid, and mixtures of these diols and acids.

In yet another aspect, the invention generally relates to a method for preparing p-phenylenediamine comprising amination of N-(4-hydroxyphenyl)ethanamide. In certain embodiments, the amination of N-(4-hydroxyphenyl)ethanamide is carried out in the presence of a precious metal catalyst on a support.

Method for the Biological Production of PABA, p-Aminophenol and N-(4-Hydroxyphenyl)Ethanamide in E. coli:

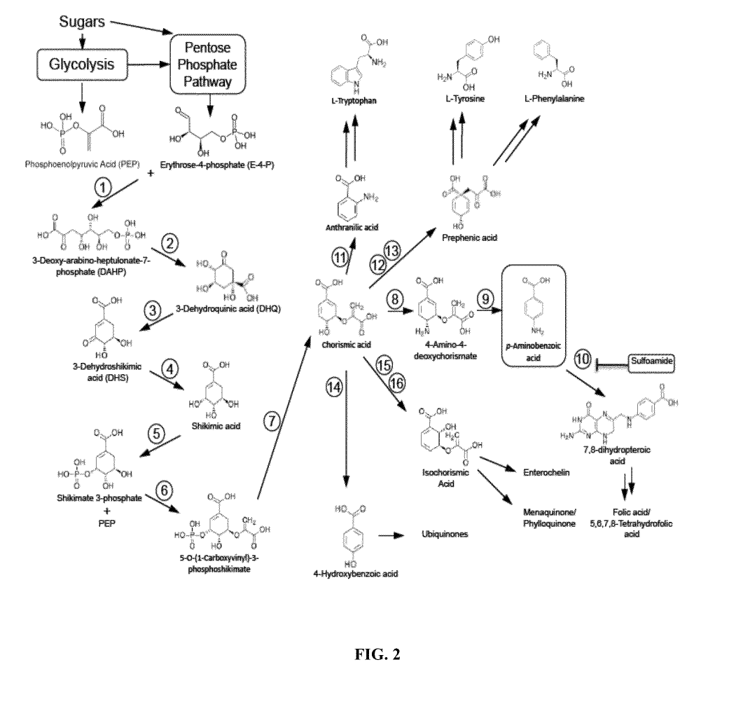

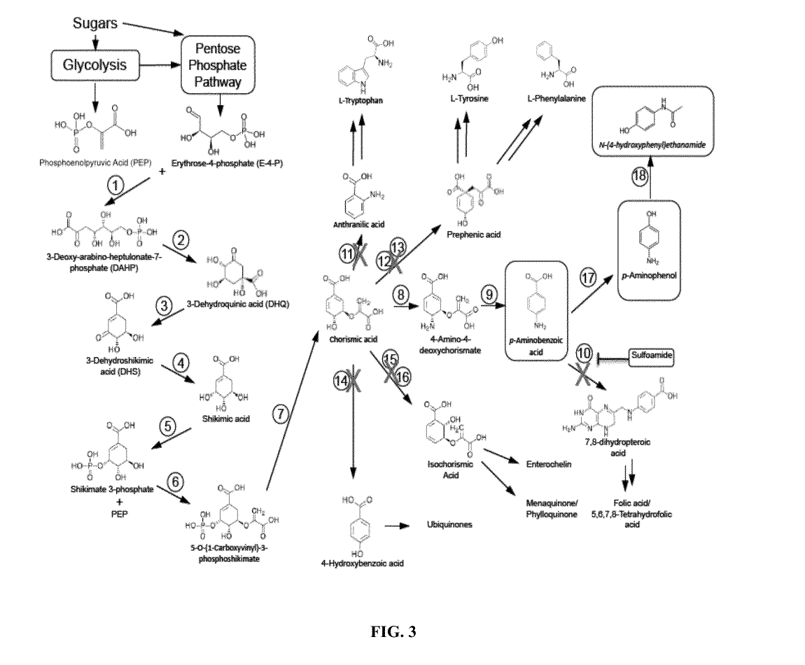

The metabolic pathway for production PABA in E. coli is outlined in FIGS. 2 and 3. The native shikimic acid pathway is shown in FIG. 2 including the condensation of phosphoenolpyruvate (PEP) and erythrose-4-phosphate (E-4-P) to the aromatic amino acids (tryptophan, tyrosine and phenylalanine) From chorismic acid, a branch of the shikimic acid pathway leads to the formation of PABA and ultimately folic acid and tetrahydrofolic acid.

FIG. 2 shows the shikimic acid pathway in E. coli. Key metabolites of the pathway are shown. Enzymatic steps and corresponding genes (Enzyme/Gene) are represented by numbers:
(1) 3-Deoxy-D-arabino-heptulosonate-7-phosphate (DAHP) synthase (EC:2.5.1.54)/aroF, aroG, aroH;
(2) 3-Dehydroquinate synthase (EC:4.2.3.4)/aroB;
(3) 3-dehydroquinate dehydratase (EC:4.2.1.10)/aroD;
(4) Dehydroshikimate reductase, NAD(P)-binding (EC:1.1.1.25)/aroE and Quinate/Shikimate 5-dehydrogenase, NAD(P)-binding (EC:1.1.1.25)/ydiB;
(5) Shikimate kinase (E.C.:2.7.1.71)/aroL;
(6) 5-Enolpyruvylshikimate-3-phosphate (EPSP) synthetase (EC:2.5.1.19)/aroA;
(7) Chorismate synthase (EC:4.2.3.5)/aroC;
(8) Aminodeoxychorismate synthase (EC:2.6.1.85)/pabA, pabB;
(9) 4-amino-4-deoxychorismate lyase component of para-aminobenzoate synthase multienzyme complex (EC:4.1.3.38)/pabC;
(10) 7,8-Dihydropteroate synthase (EC:2.5.1.15)/folP;
(11) Anthranilate synthase/anthranilate phosphoribosyl transferase (EC:4.1.3.27, EC: 2.4.2.18)/trpED;
(12) Fused chorismate mutase P/prephenate dehydratase (EC:5.4.99.5, EC:4.2.1.51)/pheA;
(13) Fused chorismate mutase P/prephenate dehydratase (EC:5.4.99.5, EC:4.2.1.51)/tyrA;
(14) Chorismate-pyruvate lyase (EC:4.1.3.40)/ubiC;
(15) Isochorismate synthase 1 (EC:5.4.4.2)/entC;
(16) Isochorismate synthase 1 (EC:5.4.4.2)/menF.

Methods and composition of the invention relate to reconfiguration of the shikimic acid pathway to produce PABA is to enhance the biosynthesis of PABA by reducing the carbon flux to the folate and other competing pathways. To produce PABA, the immediate enzymatic step after PABA, the 7,8-dihyropteroate synthase (Step 10; corresponding to genes folP), is inactivated either by mutation or enzymatic inhibitors (FIG. 3).

Gene inactivation is accomplished via allelic exchange as described before. (Link, et al. 1997 “Methods for Generating Precise Deletions and Insertions in the Genome of Wild-Type Escherichia coli: Application to Open Reading Frame Characterization” Journal Of Bacteriology 179: 6228-6237.) Alternatively, enzymatic activity of 7,8-dihyropteroate synthase can be inhibited by the addition of a sulfonamide in the culture medium. In either case, the resulting mutant or chemically treated host cell is expected to accumulate PABA. This PABA deficient mutant lacks the ability to synthesize the essential folic acid and 5,6,7,8-tetrahydrofolic acid and requires the following supplementations for proper growth: methionine, glycine, thymidine, and pantothenate. (Singer, et al. 1985 “Isolation Of A Dihydrofolate Reductase-Deficient Mutant Of Escherichia-Coli” Journal Of Bacteriology 164(1): 470-472.) Direct folic acid supplementation to wildtype E. coli is not feasible since wildtype cells lack the necessary transporter for folic acid uptake. To ameliorate the folic acid transport deficiency in the 7,8-dihyropteroate synthase-deficient mutant, folic acid transporter from Arabidopsis thaliana or Synechocystis sp. PCC6803 is introduced to E. coli (Klaus, et al. 2005 “Higher Plant Plastids And Cyanobacteria Have Folate Carriers Related To Those Of Trypanosomatids” Journal Of Biological Chemistry 280(46): 38457-38463). The resulting E. coli strain can grow in minimal medium in the presence of (6R,6S)-5-formyl-tetrahydrofolic acid or folic acid.

The carbon flux towards PABA is increased by the overexpression of aminodeoxychorismate synthase (EC:2.6.1.85) genes, pabA and pabB and 4-amino-4-deoxychorismate lyase (EC:4.1.3.38) gene, pabC. Regulated expression of genes of interest is accomplished using defined expression systems as described. (Sorensen, et al. 2005 “Advanced genetic strategies for recombinant protein expression in Escherichia coli” Journal of Biotechnology 115: 113-128.) The increase in expression of the pabA, pabB and pabC will lead to increase in the total enzymatic activities of aminodeoxychorismate synthase and 4-amino-4-deoxychorismate lyase, which in turn increase the conversion of chorismic acid to PABA.

Alternatively, the enzymatic activities of aminodeoxychorismate synthase (EC:2.6.1.85) and 4-amino-4-deoxychorismate lyase (EC:4.1.3.38) can potentially be substituted with those of a similar enzyme complex, anthranilate synthase (EC:4.1.3.27). Unlike aminodeoxychorismate synthase (EC:2.6.1.85) and 4-amino-4-deoxychorismate lyase (EC:4.1.3.38) which catalyses the para-addition, anthranilate synthase (EC:4.1.3.27) catalyses the ortho-addition of the amine group in anthranilate. To alter the enzymatic activity of anthranilate synthase (EC:4.1.3.27), the gene (trpEDG) coding for the enzyme complex is mutated by random mutagenesis (Primrose, S. B., R. M. Twyman, 2006 “Changing genes: site-directed mutagenesis and protein engineering” In: Principles of gene manipulation and genomics, 7^{th} Edition. Pages 141-156).

PABA inhibits growth of bacteria and fungi. (Reed, et al. 1959 “Inhibition of S. cerevisiae by p-Aminobenzoic Acid and Its Reversal by the Aromatic Amino Acids” Journal of Biological Chemistry 234: 904-908.) This inhibitory effect on cell growth needs to be overcome for the production of PABA at higher concentration. The biological basis for the growth inhibition by PABA is incomplete, but experimental results suggested that addition of metabolites, such as p-hydroxybenzoic acid for E. coli or aromatic amino acids for yeast, in the shikimic acid pathway could partially relieve the growth inhibition. In addition to adding known chemical(s) to restore growth, tolerance of host cells to PABA can be increased by directed evolution. Wild-type host cells are exposed to successively higher concentrations of PABA over time. This can be done with or without mutagenesis of the original host cell population. Cells with mutation(s) that allow them to grow faster in the presence of PABA can be selected for over time. Clonal variants with high tolerance to PABA can be selected and characterized. Elite variants with favorable growth characteristics can be used as hosts for PABA production.

In addition to the 7,8-dihyropteroate synthase, any or all of the three enzymes (Steps 11, 12, 13; anthranilate synthase and chorismate mutase/prephenate dehydratase; corresponding to genes trpD, pheA, tyrA) (FIG. 3) responsible for the conversion of chorismic acid to the three aromatic amino acids can be inactivated to redirect the metabolic flux towards PABA (FIG. 3). This reduces the consumption of chorismic acid for the production of aromatic amino acids and allows this key intermediate for the production of PABA. The resulting mutant requires the supplementation of the corresponding amino acids, namely tryptophan, tyrosine or phenylalanine to restore proper growth.

The remaining enzymatic activities (Steps 14, 15, 16; corresponding to genes ubiC, entC, menF) (FIG. 3) can be inactivated by allelic exchange as described above to eliminate the loss of chorismic acid to other metabolites. For the ubiC mutation, 4-hydroxybenzoic acid is added as a supplement to maintain the viability of the mutant. (Lawrence, et al. 1974 “Biosynthesis Of Ubiquinone In Escherichia-Coli-K-12—Biochemical And Genetic Characterization Of A Mutant Unable To Convert Chorismate Into 4-Hydroxybenzoate” Journal Of Bacteriology 118(1): 41-45.) No known supplementation is needed for the entC and menF mutants (Muller, et al. 1996 “An Isochorismate Hydroxymutase Isogene In Escherichia Coli.” FEBS Letters 378(2): 131-134).

In addition to inactivation of enzymatic activities, over-expression of enzymatic activities for the synthesis of PABA is needed:
Overexpression of genes coding for the enzymes aminodeoxychorismate synthase and 4-amino-4-deoxychorismate lyase;
Overexpression of genes coding for the enzymes DAHP synthase and dehydroquinate synthase or any remaining enzymes (Steps 2-7, FIG. 2) in the shikimic acid pathway, which increases the metabolic flux into the shikimic acid pathway;
Overexpression of genes coding for the enzymes transketolase (TktA) and PEP synthase (PpsA) to increase the availability of erythose-4-phosphate and PEP respectively.

FIG. 3 shows the modified shikimic acid pathway for the production of PABA in E. coli. Key metabolites of the pathway are shown. Crosses indicate inactivation of enzymatic steps. Enzymatic steps and corresponding genes (Enzyme/Gene) are represented by numbers:
(1) 3-Deoxy-D-arabino-heptulosonate-7-phosphate (DAHP) synthase (EC:2.5.1.54)/aroF, aroG, aroH;
(2) 3-Dehydroquinate synthase (EC:4.2.3.4)/aroB;
(3) 3-dehydroquinate dehydratase (EC:4.2.1.10)/aroD;
(4) Dehydroshikimate reductase, NAD(P)-binding (EC:1.1.1.25)/aroE and Quinate/Shikimate 5-dehydrogenase, NAD(P)-binding (EC:1.1.1.25)/ydiB;
(5) Shikimate kinase (E.C.:2.7.1.71)/aroL;
(6) 5-Enolpyruvylshikimate-3-phosphate (EPSP) synthetase (EC:2.5.1.19)/aroA;
(7) Chorismate synthase (EC:4.2.3.5)/aroC;
(8) Aminodeoxychorismate synthase (EC:2.6.1.85)/pabA, pabB;
(9) 4-amino-4-deoxychorismate lyase component of para-aminobenzoate synthase multienzyme complex (EC:4.1.3.38)/pabC;
(10) 7,8-Dihydropteroate synthase (EC:2.5.1.15)/folP;
(11) Anthranilate synthase/anthranilate phosphoribosyl transferase (EC:4.1.3.27, EC: 2.4.2.18)/trpED;
(12) Fused chorismate mutase P/prephenate dehydratase (EC:5.4.99.5, EC:4.2.1.51)/pheA;
(13) Fused chorismate mutase P/prephenate dehydratase (EC:5.4.99.5, EC:4.2.1.51)/tyrA;
(14) Chorismate-pyruvate lyase (EC:4.1.3.40)/ubiC;
(15) Isochorismate synthase 1 (EC:5.4.4.2)/entC;
(16) Isochorismate synthase 1 (EC:5.4.4.2)/menF.
(17) 4-aminobenzoate 1-monooxygenase (EC:1.14.13.27);
(18) Arylamine N-acetyltransferases (EC:2.3.1.5).

PABA can be enzymatically converted to p-aminophenol by 4-aminobenzoate 1-monooxygenase (EC:1.14.13.27). For example, 4-aminobenzoate 1-monooxygenase from Agaricus biosporus was shown to be effective in the conversion in vitro (Tsuji et al, 1985 “A unique enzyme catalyzing the formation of 4-hydroxyaniline from 4-amino-benzoic acid in Agaricus bisporus.” Biochem. Biophys Res Commun. 130(2):633-639. Tsuji et al. 1986 “Purification and properties of 4-aminobenzoate hydroxylase, a new monooxygenase from Agaricus bisporus.” J Biol Chem 261(28):13203-13209. Tsuji et al. 1996 “Cloning and sequencing of cDNA encoding 4-aminobenzoate hydroxylase from Agaricus bisporus.” Biochim Biophys Acta 1309(1-2):31-36.).

p-Aminophenol can be further converted enzymatically to N-(4-hydroxyphenyl)ethanamide by arylamine N-acetyltransferases (EC:2.3.1.5) (Mulyono et al. 2007 “Bacillus cereus strain 10-L-2 produces two arylamine N-acetyltransferases that transform 4-phenylenediamine into 4-aminoacetanilide.”J Biosci Bioeng 103(2):147-154.) Different arylamine N-acetyltransferases have different substrate specificity. The NAT-a enzyme from Bacillus cereus strain 10-L-2 was shown to have a higher selectivity for p-aminophenol than NAT-b.

Method for the Biological Production of PABA, p-Aminophenol and N-(4-Hydroxyphenyl)Ethanamide in S. cerevisiae:

Metabolic pathway engineering involves, for example,
constructing a S. cerevisiae strain with a mutation in FOL1, blocking further conversion of PABA to folic acid. Eliminate competing pathways for chorismic acid by the inactivation of ARO7, TRP2, and PHA2 genes, singly or in combination;
producing S. cerevisiae vectors for the overexpression of biosynthetic enzymes for the conversion of chorismic acid to PABA: aminodeoxychorismate synthase (ABZ1) and 4-amino-4-deoxychorismate lyase (ABZ2).
producing S. cerevisiae vectors for the expression of a DAHP synthase isozyme aroFFBR from E. coli that is insensitive to feedback inhibition by tyrosine and other aromatic amino acids.

Two methods can be exploited for construction of the mutants: (1) construction of all mutants de novo, and (2) mating of previously characterized mutants. Both options are viable, but construction of de novo mutants will probably be faster than mating, so we will start there and follow-up with a crossing strategy. The initial step involves construction of a fol1Δ strain deficient in 7,8-dihydropteroate synthase activity, blocking further assimilation of PABA into folic acid. The resulting fol1Δ mutant requires 5,6,7,8-tetrahydrofolic acid supplementation to maintain growth. Media modification with aromatic amino acids and higher pH may also be necessary if a high concentration of PABA is produced. (Krömer, et al. 2012 J. Biotech. in press.) To the fol1Δ background, additional mutations (aro7Δ, trp2Δ, and pha2Δ) will be added to eliminate competing pathways for chorismic acid, either singly or in combination. Some combination of mutations may result in poor growth or lethality due to high-level production of PABA. In those cases, a knockdown approach in which the wild-type gene is replaced with a mutated, less active gene is preferable. The mutated gene replacement creates a partial block, but still allows some carbon flux through the competing pathway. When a highly PABA-tolerant host strain can be obtained from the direct selection, the pathway design can be transferred to the tolerant host, tested and optimized further.

De novo mutant construction can be performed with S. cerevisiae strain BY4741 (MATa his3Δ1 leu2Δ0 met15Δ0 ura3Δ0), a widely used strain, which conveniently has four auxotrophic markers that can be exploited for selection to prototrophy. Three of the four target loci (ARO7, TRP2, and PHA2) will be inactivated by insertion of an expressed version of LEU2, MET15 and URA3, followed by selection for prototrophy. To maintain his3Δ1 for plasmid selection, a kanamycin-resistance cassette will be used to inactivate FOL1. Inactivation in this manner is a rapid technique that can be performed serially to generate the necessary strains. (Hegemann, et al. Gene Disruption in the Budding Yeast Saccharomyces cerevisiae, 2005. p. 129-144.)

For mating of mutants, mutants from the Yeast Deletion Collection can be obtained. (e.g., Available from: http://clones.invitrogen.com/cloneinfo.php?clone=yeast.) The following table shows data for each of the necessary strains.

TABLE 1

| | Record | | | | Parent |
|---|---|---|---|---|---|
| Gene Name | No. | ORF Name | Mating Types | Ploidy | Strain |
| TRP2 | 6395 | YER090W | MATa | Haploid | BY4741 |
| ARO7 | 5479 | YPR060C | MATa | Haploid | BY4741 |
| PHA2 | 6472 | YNL316C | MATa | Haploid | BY4741 |
| TRP2 | 16395 | YER090W | MATa | Haploid | BY4742 |
| ARO7 | 15479 | YPR060C | MATa | Haploid | BY4742 |
| PHA2 | 16472 | YNL316C | MATa | Haploid | BY4742 |
| FOL1 | 26466 | YNL256W | MATa/α | Diploid | BY4743 |

The mutants were originally constructed in BY4743 (A derivative of BY4741; MATa/a his3D1/his3D1 leu2D0/leu2D0 lys2D0/LYS2 MET15/met15D0 ura3D0/ura3D0) and sporulated to produce haploids when possible. Note that ARO7, TRP2 and PHA2 are available as haploids, but that FOL1 must be obtained as a heterozygous diploid due to its folate auxotrophy. This strain can be grown and sporulated under folate supplementation to provide the appropriate haploid strain for mating. Mating can be performed according standard protocols. (Guthrie, C., Guide to Yeast Genetics and Molecular Biology. Methods in Enzymology, ed. C. Guthrie and G. R. Fink. Vol. 350. 1991: Academic Press. 623.) The resulting strains will be kanamycin resistant due to the insertions at each mutant locus.

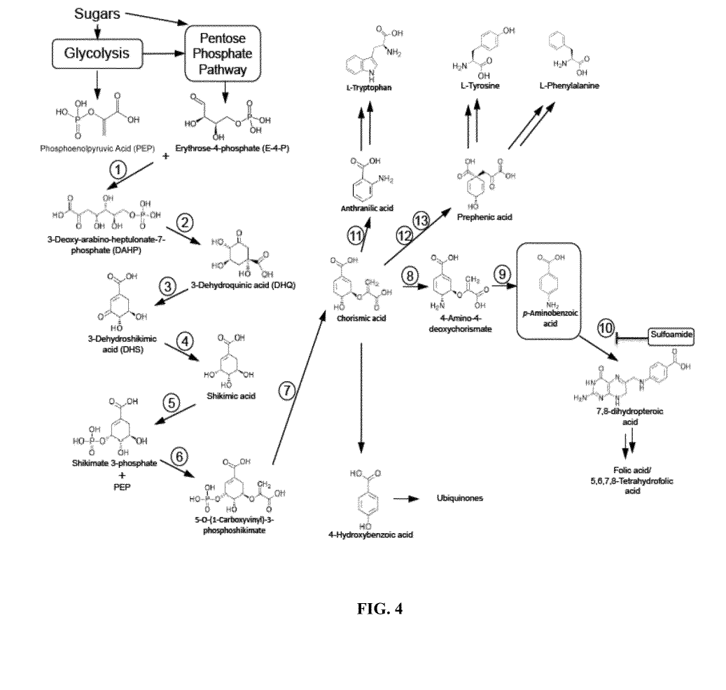

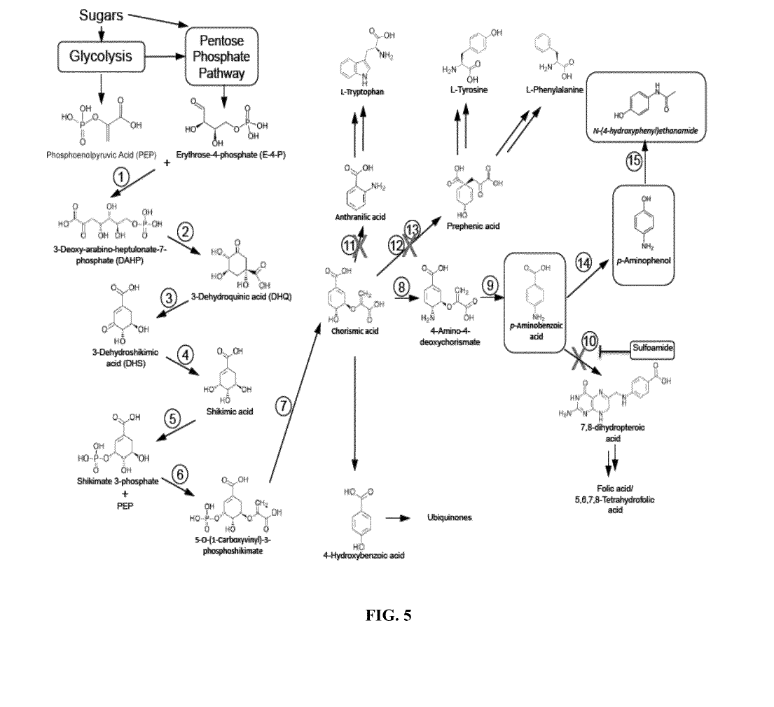

The metabolic pathway for production PABA in S. cerevisiae is outlined in FIGS. 4 and 5. The native shikimic acid pathway is shown in FIG. 4 including the condensation of phosphoenolpyruvate (PEP) and erythrose-4-phosphate (E-4-P) to the aromatic amino acids (tryptophan, tyrosine and phenylalanine) From chorismic acid, a branch of shikimic acid pathway leads to the formation of PABA and ultimately folic acid and tetrahydrofolic acid. Unlike E. coli, in yeast, the enterchelin and menaquinone/phylloquinone pathways are absent.

FIG. 4 shows the shikimic acid pathway in S. cerevisiae. Key metabolites of the pathway are shown. Enzymatic steps and corresponding genes (Enzyme/Gene) are represented by numbers:
(1) 3-Deoxy-D-arabino-heptulosonate-7-phosphate (DAHP) synthase (EC:2.5.1.54)/ARO4;
(2) Pentafunctional AROM polypeptide (EC:2.7.1.71, EC: 1.1.1.25, EC:2.5.1.19, EC:4.2.3.4, EC: 4.2.1.10))/ARO1;
(3) Pentafunctional AROM polypeptide (EC:2.7.1.71, EC: 1.1.1.25, EC:2.5.1.19, EC:4.2.3.4, EC: 4.2.1.10))/ARO1;
(4) Pentafunctional AROM polypeptide (EC:2.7.1.71, EC: 1.1.1.25, EC:2.5.1.19, EC:4.2.3.4, EC: 4.2.1.10))/ARO1;
(5) Pentafunctional AROM polypeptide (EC:2.7.1.71, EC: 1.1.1.25, EC:2.5.1.19, EC:4.2.3.4, EC: 4.2.1.10))/ARO1;
(6) Pentafunctional AROM polypeptide (EC:2.7.1.71, EC: 1.1.1.25, EC:2.5.1.19, EC:4.2.3.4, EC: 4.2.1.10))/ARO1;
(7) Chorismate synthase (EC:4.2.3.5)/ARO2;
(8) Aminodeoxychorismate synthase (EC:2.6.1.85)/ABZ1;
(9) 4-amino-4-deoxychorismate lyase component of para-aminobenzoate synthase multienzyme complex (EC:4.1.3.38)/ABZ2;
(10) Dihydroneopterin aldolase/2-amino-4-hydroxy-6-hydroxymethyldihydropteridine diphosphokinase/dihydropteroate synthase (EC:4.1.2.25, EC:2.7.6.3, EC:2.5.1.15)/FOL1;
(11) Anthranilate synthase component I and II (EC:4.1.3.27)/TRP2 and TRP3;
(12) Fused chorismate mutase P/prephenate dehydratase (EC:5.4.99.5, EC:4.2.1.51)/PHA2;
(13) Fused chorismate mutase P/prephenate dehydratase (EC:5.4.99.5, EC:4.2.1.51)/ARO7.

To produce PABA, the immediate enzymatic step after PABA, the 7,8-dihyropteroate synthase (Step 10; corresponding to gene FOL1), is inactivated either by mutation or enzymatic inhibitors (FIG. 5). Gene inactivation can be accomplished via allelic exchange as described. (Klinner, et al. 2004 “Genetic aspects of targeted insertion mutagenesis in yeasts” FEMS Microbiology Reviews 28 (2004) 201-223.) Alternatively, enzymatic activity of 7,8-dihyropteroate synthase can be inhibited by the addition of a sulfonamide in the culture medium. The resulting mutant is expected to accumulate PABA. This PABA deficient mutant lacks the ability to synthesize the essential folic acid and 5,6,7,8-tetrahydrofolic acid and requires the supplementation of 5-formyl tetrahydrofolic acid for proper growth (Guldener, et al. 2004 “Characterization Of The Saccharomyces cerevisiae Foll Protein: Starvation For C1 Carrier Induces Pseudohyphal Growth” Molecular Biology Of The Cell 15(8): 3811-3828).

In addition to the 7,8-dihyropteroate synthase, any of the three enzymes (Steps 11, 12, 13; anthranilate synthase and chorismate mutase/prephenate dehydratase; corresponding to genes TRP2 and TRP3, PHA2, ARO7) (FIG. 5) responsible for the conversion of chorismic acid to the three aromatic amino acids are inactivated to redirect the metabolic flux towards PABA (FIG. 5). The resulting mutant will require the supplementation of the corresponding amino acids tryptophan, tyrosine and phenylalanine to restore proper growth.

For example, the aminodeoxychorismate synthase (pabA and pabB) and 4-amino-4-deoxychorismate lyase (pabC) activities may be increased by the overexpression of the corresponding genes, which enhance the conversion of chorismic acid to PABA. Alternatively, gene fusions between pabA and pabB (pabAB) as found in actinomyces, Plasmodium falciparum, and Arabidopsis thaliana may be employed in place of pabA and pabB. (James, et al. 2002 “The Pabl Gene Of Coprinus Cinereus Encodes A Bifunctional Protein For Para-Aminobenzoic Acid (PABA) Synthesis: Implications For The Evolution Of Fused PABA Synthases” Journal Of Basic Microbiology 42(2): 91-103; Basset, et al. 2004 “Folate Synthesis In Plants: The Last Step Of The P-Aminobenzoate Branch Is Catalyzed By A Plastidial Aminodeoxychorismate Lyase” Plant Journal 40(4): 453-461.)

In another example, gene fusion between pabB and pabC (pabBC), as found in Lactococcus lactis, can be used in place of the pabB and pabC genes. (Wegkamp, et al. 2007 “Characterization Of The Role Of Para-Aminobenzoic Acid Biosynthesis In Folate Production By Lactococcus Lactis” Applied And Environmental Microbiology 73(8): 2673-2681.)

The mutation(s) may confer only partial inactivation of enzymatic activities.

Any or all of the following competing pathways for chorismic acid may be inactivated by mutations or enzyme inhibitors.
(11) Anthranilate synthase component I and II (EC:4.1.3.27)/TRP2 and TRP3;
(12) Fused chorismate mutase P/prephenate dehydratase (EC:5.4.99.5, EC:4.2.1.51)/PHA2;
(13) Fused chorismate mutase P/prephenate dehydratase (EC:5.4.99.5, EC:4.2.1.51)/ARO7.

The mutation(s) may confer only partial inactivation of enzymatic activities.

The mutants may require specific supplemental metabolites to maintain cell viability: Tryptophan for (11) Anthranilate synthase component I and II (EC:4.1.3.27)/TRP2 and TRP3; Phenylalanine for (12) Fused chorismate mutase P/prephenate dehydratase (EC:5.4.99.5, EC:4.2.1.51)/PHA2; Tyrosine for (13) Fused chorismate mutase P/prephenate dehydratase (EC:5.4.99.5, EC:4.2.1.51)/ARO7.

The carbon flux towards PABA is increased by the overexpression of aminodeoxychorismate synthase (EC:2.6.1.85) gene, ABZ1 and 4-amino-4-deoxychorismate lyase (EC:4.1.3.38) gene, ABZ2. Regulated expression of genes of interest can be accomplished using defined expression systems as described (Michael, et al. 1992 “Foreign Gene Expression in Yeast: a Review” YEAST 8: 423-488). The increase in expression of the ABZ1 and ABZ2 leads to an increase in the total enzymatic activities of aminodeoxychorismate synthase and 4-amino-4-deoxychorismate lyase, which in turn increase the conversion of chorismic acid to PABA.

Alternatively, the enzymatic activities of aminodeoxychorismate synthase (EC:2.6.1.85) and 4-amino-4-deoxychorismate lyase (EC:4.1.3.38) can potentially be substituted with those of a similar enzyme complex, anthranilate synthase (EC:4.1.3.27). Unlike aminodeoxychorismate synthase (EC:2.6.1.85) and 4-amino-4-deoxychorismate lyase (EC:4.1.3.38) which catalyses the para-addition, anthranilate synthase (EC:4.1.3.27) catalyses the ortho-addition of the amine group in anthranilate. To alter the enzymatic activity of anthranilate synthase (EC:4.1.3.27), the genes (trpEDG) coding for the enzyme complex is mutated by random mutagenesis. (Primrose, et al. 2006 “Changing genes: site-directed mutagenesis and protein engineering” In: Principles of gene manipulation and genomics, 7^{th} Edition. Pages 141-156.)

PABA inhibits growth of bacteria and fungi (Reed, et al. 1959 “Inhibition of Saccharomyces cerevisiae by p-Aminobenzoic Acid and Its Reversal by the Aromatic Amino Acids” Journal of Biological Chemistry 234: 904-908). This inhibitory effect on cell growth needs to be overcome for the production of PABA at higher concentration. The biological basis for the growth inhibition by PABA is incomplete, but experimental results suggested that addition of metabolites, such as p-hydroxybenzoic acid for E. coli or aromatic amino acids for yeast, in the shikimic acid pathway could partially relieve the growth inhibition. In addition to added known chemical(s) to restore growth, tolerance of host cells to PABA can be increased by directed evolution. Wild-type host cells are exposed to successive higher concentrations of PABA over time. This can be done with or without mutagenesis of the original host cell population. Cells with mutation(s) that allow them to grow faster in the presence of PABA will be selected for over time. Clonal variants with high tolerance to PABA will be selected and characterized. Elite variants with favorable growth characteristics will be used as hosts for PABA production.

Furthermore, in a strategy that can help both with PABA resistance and continuous PABA fermentation, multidrug efflux pumps can be utilized to pump PABA out of the cell as it is produced. Sulfonamide antibiotics are PABA analogs, and resistance can be achieved via efflux pumps. (Alekshun, et al. 2007 Cell. 128(6): p. 1037-1050.) PABA exporters can be produced by targeted modification or directed evolution for PABA tolerance. A similar procedure can be made for export of p-aminophenol.

In addition to inactivation of enzymatic activities, over-expression of enzymatic activities for the synthesis of PABA is needed:
Overexpression of genes coding for the enzymes aminodeoxychorismate synthase (corresponding to the gene ABZ1) and PABA synthase (corresponding to the gene ABZ2). Overexpression of genes in yeast can be achieved as described previously;
Overexpression of genes coding for the enzymes DAHP synthase and the AROM protein in the pathway, which increases the metabolic flux into the shikimic acid pathway;
Overexpression of genes coding for the enzymes transketolase (TKL1) and PEP synthase (PpsA) to increase the availability of erythose-4-phosphate and PEP respectively. (Sundstrom, et al. 1993 “Yeast TKLI Gene Encodes A Transketolase That Is Required For Efficient Glycolysis And Biosynthesis Of Aromatic Amino Acids” Journal Of Biological Chemistry 268(32): 24346-24352.)

FIG. 5 shows the modified shikimic acid pathway for the production of PABA in S. cerevisiae. Key metabolites of the pathway are shown. Crosses indicate inactivation of enzymatic steps. Enzymatic steps and corresponding genes (Enzyme/Gene) are represented by numbers:
(1) 3-Deoxy-D-arabino-heptulosonate-7-phosphate (DAHP) synthase (EC:2.5.1.54)/ARO4;
(2) pentafunctional AROM polypeptide (EC:2.7.1.71, EC: 1.1.1.25, EC:2.5.1.19, EC:4.2.3.4, EC: 4.2.1.10))/ARO1;
(3) pentafunctional AROM polypeptide (EC:2.7.1.71, EC: 1.1.1.25, EC:2.5.1.19, EC:4.2.3.4, EC: 4.2.1.10))/ARO1;
(4) pentafunctional AROM polypeptide (EC:2.7.1.71, EC: 1.1.1.25, EC:2.5.1.19, EC:4.2.3.4, EC: 4.2.1.10))/ARO1;
(5) pentafunctional AROM polypeptide (EC:2.7.1.71, EC: 1.1.1.25, EC:2.5.1.19, EC:4.2.3.4, EC: 4.2.1.10))/ARO1;
(6) pentafunctional AROM polypeptide (EC:2.7.1.71, EC: 1.1.1.25, EC:2.5.1.19, EC:4.2.3.4, EC: 4.2.1.10))/ARO1;
(7) Chorismate synthase (EC:4.2.3.5)/ARO2;
(8) Aminodeoxychorismate synthase (EC:2.6.1.85)/ABZ1;
(9) 4-amino-4-deoxychorismate lyase component of para-aminobenzoate synthase multienzyme complex (EC:4.1.3.38)/ABZ2;
(10) Dihydroneopterin aldolase/2-amino-4-hydroxy-6-hydroxymethyldihydropteridine diphosphokinase/dihydropteroate synthase (EC:4.1.2.25, EC:2.7.6.3, EC:2.5.1.15)/FOL1;
(11) Anthranilate synthase component I and II (EC:4.1.3.27)/TRP2 and TRP3;
(12) Fused chorismate mutase P/prephenate dehydratase (EC:5.4.99.5, EC:4.2.1.51)/PHA2;
(13) Fused chorismate mutase P/prephenate dehydratase (EC:5.4.99.5, EC:4.2.1.51)/ARO7;
(14) 4-aminobenzoate 1-monooxygenase (EC:1.14.13.27);
(15) Arylamine N-acetyltransferases (EC:2.3.1.5).
   S. cerevisiae Vectors

S. cerevisiae vectors for the overexpression of biosynthetic enzymes for the conversion of chorismic acid to PABA can be aminodeoxychorismate synthase (ABZ1) and 4-amino-4-deoxychorismate lyase (ABZ2), singly and in combination.

S. cerevisiae/E. coli shuttle vector pRS423 can be used, which contains a 2μ origin and the yeast HIS3 selectable marker. (Christianson, et al. 1992 Gene 110(1): p. 119-22.) This plasmid can be used in the strains, as all will contain his3Δ1. Vectors can be constructed that express each gene individually, and the two in combination. Promoter/terminator combinations can be selected from among TEF2, PYK1, and ENO2. (Sun, et al., 2012 Biotechnol. Bioeng. 109, 8, p. 2082-92.) Each Promoter/ORF/Terminator combination can be designed, synthesized commercially, and subcloned into pRS423. The resulting vectors can be used to transform the appropriate S. cerevisiae mutant strains to overexpress aminodeoxychorismate synthase (ABZ1) and 4-amino-4-deoxychorismate lyase (ABZ2), singly and in combination. (Hinnen, et al. 1978 Proc Natl Acad Sci USA 75(4): p. 1929-33.) Functionality of clones can be assayed enzymatically. (Tsuji, et al., 1985 Biochem. & Biophys. Res. Comm. 130(2): p. 633-639; Tsuji, H., et al., 1986 J. Biol. Chem. 261(28): p. 13203-9; Brooke, et al., 2003 Bioorg. Med. Chem. 11(7): p. 1227-34.)

S. cerevisiae vectors for the expression of a DAHP synthase isozyme aroF^{FBR} from E. coli can be produced that are insensitive to feedback inhibition by tyrosine and other aromatic amino acids. S. cerevisiae integration or plasmid expression vector can be constructed to express the heterologous DAHP synthase isozyme aroF^{FBR} from E. coli in the yeast host strain. (Weaver, et al. 1990 J. Bacteriol. 172(11): p. 6581-4.) DAHP synthase catalyses the committing step in the shikimic acid pathway and is subject to feedback inhibition by aromatic amino acids. (Helmstaedt, et al. 2005 Proc Natl Acad Sci USA 102(28): p. 9784-9.) The yeast DAHP synthase isozymes ARO3 and ARO4 are feedback inhibited by phenylalanine and tyrosine respectively. In the presence of supplemental aromatic amino acids, the carbon flux through the shikimic acid will be restricted due to the feedback inhibition on ARO3 and ARO4. The expression of the feedback resistant aroF^{FBR} can circumvent the inhibition.

S. cerevisiae strains resistant to the targeted metabolites can be selected. The target molecules are all inhibitors of wild-type S. cerevisiae, which may limit our ability to overproduce these molecules. (Brennan, et al. 1997 Mutagenesis 12(4): p. 215-20; Srikanth, et al. 2005 Microbiology 151(Pt 1): p. 99-111.) For example, the solubility of PABA in water is 0.072 M (about 10 g/L), and the most PABA-tolerant strains identified to date tolerates less than one fifth of that concentration. (Bradley, et al., Open Notebook Science Challenge: Solubilities of Organic Compounds in Organic Solvents Nature Precedings, 2010 http://dx.doi.org/10.1038/npre.2010.4243.3; Krömer, et al., 2012 Production of aromatics in Saccharomyces cerevisiae—A feasibility study. Journal of Biotechnology.) Selection of resistant strains can be initiated by growing mutant strains in media containing increasing amounts of each molecule to derive resistant strains. A large bank of wild-type S. cerevisiae strains can be screened.

Production of p-Aminophenol from PABA

PABA can be enzymatically converted to p-aminophenol by 4-aminobenzoate 1-monooxygenase (EC:1.14.13.27). For example, 4-aminobenzoate 1-monooxygenase from Agaricus biosporus was shown to be effective in the conversion in vitro (Tsuji et al. 1985 “A unique enzyme catalyzing the formation of 4-hydroxyaniline from 4-amino-benzoic acid in Agaricus bisporus.” Biochem Biophys Res Commun. 130(2):633-639. Tsuji et al, 1986 “Purification and properties of 4-aminobenzoate hydroxylase, a new monooxygenase from Agaricus bisporus.” J Biol Chem 261(28):13203-13209. Tsuji et al, 1996 “Cloning and sequencing of cDNA encoding 4-aminobenzoate hydroxylase from Agaricus bisporus.”Biochim Biophys Acta 1309(1-2):31-36.).

p-Aminophenol can be further converted enzymatically to N-(4-hydroxyphenyl)ethanamide by arylamine N-acetyltransferases (EC:2.3.1.5) (Mulyono et al. 2007 “Bacillus cereus strain 10-L-2 produces two arylamine N-acetyltransferases that transform 4-phenylenediamine into 4-aminoacetanilide.” J Biosci Bioeng 103(2):147-154.) Different arylamine N-acetyltransferases have different substrate specificity. The NAT-a enzyme from Bacillus cereus strain 10-L-2 was shown to have a higher selectivity for p-aminophenol than NAT-b.

Production of p-Phenylenediamine (PPD) from p-Aminophenol

Also disclosed herein are novel methods to synthesize PPD. The synthesis can be accomplished in a single step.

p-Aminophenol can be converted to PPD (PPD) by amination using catalysts such as noble metal catalysts in the presence of ammonia and hydrogen. Such a conversion has been described in the art, for example by M. Yasuhara and co-workers in Japanese Patent Nos. 1988057559 and 1990069448. A list of such processes for converting aminophenols and dihydroxybenzenes to diaminobenzenes can be found in in R. S. Downing P. J Kunkler, and H. van Bekkum, Catalysis Today, 1997, Vol. 37, 121-136; see also, M. Hauptreif and H. Reichelt, EP514487; H. Oikawa, M. Ishibashi, K. Maeda, H. Tarumoto, and I Hashimoto, JP06345701(1994); Y. Watabe, Y. Naganuma, E. Sugiyama, and T. Komiyama, JP03112946(1991); and M. Yasuhara and F. Matusanaga, 02069448(1990); these references cited therein are hereby expressly incorporated herein by reference for all purposes. Such processes can be used to convert bio-para-aminophenol to bio-PPD, where the PABA is derived from fermentation of sugars as described above. Thus, the process described directly above can be employed to prepare bio-PPD from bio-PABA. Such a two-step process is shown in Example A below:

| | Conditions | | Conditions | |
|---|---|---|---|---|
| 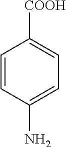 | Catalyst 100-250°C. 1-100 atm. | 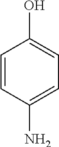 | Catalyst H₂/NH₃ | 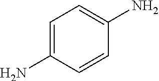 |
| | Catalyst example: CuO/ZSM-1 | | Catalyst example: Pd/C | |

Also disclosed herein is a novel method to synthesize PPD by hydrogenolysis/decarboxylation and amination of PABA acid by precious metal and base metal catalyst under the pressure of about 15 psi to about 5000 psi, preferably about 500 to about 1000 psi, and at a temperature of about room temperature to about 400° C., preferably about 150° C. to about 250° C.

Heterogeneous catalysts used for the present invention are supported on an inert carrier. The active metal component of the catalyst is selected from Ru, Pd, Pt, Rh, Re, Au, Ir, Ni, Cu, Cr, Co, or their combination. The representative carriers include activated carbon (AC), ceria (CeO₂), alumina (Al₂O₃), zirconia (ZrO₂), titania (TiO₂), silica (SiO₂) and their mixtures. The amount of precious metal and base metal catalyst for this reaction is in the range of about 0.01% to about 40% by weight based on the starting aromatic compound. The metal loading on the carrier is about 0.1% to about 60% by weight.

The hydrogenolysis/decarboxylation and amination of the present invention can be carried out either in a batch or in a continuous process in H₂ and/or NH₃ atmosphere. Total reaction time in batch reactor is about 30 to about 240 min. The longer reaction at higher temperature and under higher H₂ pressure may cause an increase in the undesirable by-products formation and saturation of aromatic ring, respectively.

Taking the reported PPD synthesis methods into consideration, the present invention provides the simple and green method of preparing PPD by heterogeneous catalytic hydrogenolysis and amination. By this invention, reaction pathway is significantly shortened and the formation of by-products, including halogenated compounds, is greatly suppressed.

Production of Aniline from PABA

It will be understood that methods disclosed herein for preparing aniline from PABA can be applied equally well to PABA derived from petroleum or biological sources. Aniline can be prepared by decarboxylation of PABA in solution in the presence of an acid catalyst. The reaction can be carried out in water as solvent containing hydrochloric acid. The reaction is typically carried out at elevated temperature to maintain a decarboxylation rate that is practical for commercial application. The preferred temperature is in the range of about 50° C. to about 100° C. and more preferably in the range of about 60° C. to about 80° C. The amount of hydrochloric acid is added that is sufficient to maintain a practical rate of decarboxylation.

In another embodiment, the reaction can be carried out at a temperature in excess of the melting point of PABA, which is about 187° C. to about 189° C. Under these conditions, the desired aniline can be removed from the reactor by distillation since the boiling point of aniline is 183° C. The reaction can be facilitated by addition of a high boiling solvent with a boiling point high enough to maintain a practical rate of decarboxylation and also in excess of the aniline boiling point to facilitate removal of aniline from the reaction. Examples of such high boiling solvents include diphenyl ether, diglycerol and triglycerol.

Examples of conversion of PABA to aniline have been reported. For example, Carstensen and Musa in J. Pharmaceutical Sciences, 1972, Vol. 61, pages 1112-1118, reported that decarboxylation of PABA gives aniline as the only product. Decarboxylation of PABA and related compounds such as anthranilic and substituted anthranilic acids have also been reported by Clark (J. Physical Chemistry, 1963, Vol. 67, 138-140) and Dunn and Prysiazniuk (Canadian J. Chemistry, 1961, Vol. 39, 285-296). None of these reports involves use of the decarboxylation as a synthetic procedure for making aniline, nor do any of the reports describe using decarboxylation of biologically-derived PABA as a synthetic route to biologically-derived aniline.

### Production of Methylenedianiline (MDA) and Methylene Diphenyl Diisocyanate (MDI)

Disclosed here are methods for producing methylenedianiline (MDA) from aniline that is made from biologically-derived PABA and MDA that is made directly from PABA (either biologically-derived or petroleum-derived). The biologically-derived MDA is useful in preparing biologically-derived methylene diphenyl diisocyanate, which in turn is useful in preparing partially biologically-derived aromatic polyurethanes and, when used with biologically-derived polyols and polyester polyols, is useful in preparing 100% biologically derived, and hence renewable, polyurethanes.

Conversion of PABA-derived aniline to MDA is accomplished by reacting aniline with formaldehyde in water in the presence of a suitable acid catalyst. While a variety of acid catalysts can be used in this process, the preferred catalyst is hydrochloric acid. Such reactions have been reported for conversion of petroleum-derived aniline to MDA (see, for example, patents U.S. Pat. Nos. 2,974,168; 2,938,054; 2,818,433; 3,476,806; 3,367,969; 6,831,192; 7,038,022B2). The aniline-formaldehyde condensation reactions can be carried out under a variety of conditions, resulting in a mixture of products that can be rich in methylenedianiline isomers, with the 4,4′-methylenedianiline predominating over the 2,4′-isomer or can be richer in higher molecular weight aniline-formaldehyde condensation products resulting from further reaction of low molecular weight condensation products such as methylenedianiline with additional aniline and formaldehyde. These higher molecular weight condensation products have more than two amine groups per molecule and can be linear or branched. The condensation chemistry of aniline with formaldehyde has been discussed in detail by Twitchett in Chemical Society Reviews, 1974, Vol. 3, 209-230, which and references cited therein are hereby expressly incorporated herein by reference for all purposes.

The invention described herein also includes a new reaction for preparing aniline-formaldehyde condensation products by reacting PABA with formaldehyde in water in the presence of an acid catalyst such as hydrochloric acid. The inventors have found that the condensation of formaldehyde with PABA occurs with decarboxylation to produce the aniline-formaldehyde condensation products. The decarboxylation may occur 1) before the condensation reaction to produce aniline, which then condenses with formaldehyde, or 2) during the reaction of formaldehyde or a PABA-formaldehyde adduct with aniline to produce a condensation product, although the exact detail is not yet known. The important aspect of this discovery is that aniline-formaldehyde condensation products of the methylenedianiline type are produced by reaction of PABA with formaldehyde in the presence of an acid catalyst. Such methylenedianiline products are useful in preparing MDI and MDI-type isocyanates that, in turn, are useful in producing technologically important polyurethanes. When the PABA used is biologically derived through processes such as fermentation of biomass, then the methylenedianiline-type products, the resulting isocyanates and polyurethanes can be either partially or 100% biologically derived, and hence 100% renewable in the same manner as described for aniline in the previous paragraph.

Exemplary methods according to the present invention are provided by the following examples. The products of the present invention can be quantitatively analyzed by HPLC, GC-MS and/or -FID.

Further detail of the present invention is described below with the following examples, which illustrate but are not intended to limit the present invention.

### Example 1

0.1 g of 5% Ru/Al₂O₃, 1.0 g of PABA, and 30 mL of DI water are placed in 75 mL high pressure Parr reactor. The reactor is sealed and then pressurized to 200 psi by H₂. The reactor is heated up to 200° C., and the temperature is maintained for 1 hour. The reaction product is obtained after the temperature reached room temperature. The aminophenol is isolated, and placed in the 75 mL high pressure Parr reactor with 0.1 g of 5% Ru/Al₂O₃ and 30 mL of DI water. The reactor is sealed and then pressurized, first, to 50 psi by NH₃ and to 200 psi by H₂. The reactor is heated up to 200° C., and the temperature is maintained for 1 hour. The reaction product is collected after the temperature reaches room temperature and analyzed by HPLC and GC.

### Example 2-7

Experiments with Raney, Cu, CuCr, Raney Ni, Ru or Pd catalyst with different solvents, different temperature and pressure is carried out in the same manner as described in Example 1. The results are shown in Table 2.

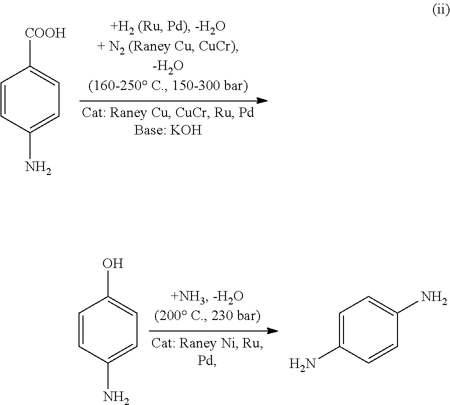 [SMILES: Nc1ccc(C(=O)O)cc1 ; Nc1ccc(O)cc1 ; Nc1ccc(N)cc1]

TABLE 2
Example 2-7

| Example | Catalyst 1 | Catalyst 2 | Solvent |
|---|---|---|---|
| 2 | Raney Cu | Raney Ni | H₂O |
| 3 | CuCr | Raney Ni | H₂O |
| 4 | Ru | Ru | H₂O |
| 5 | Ru | Ru | THF |
| 6 | Pd | Pd | H₂O |
| 7 | Pd | Pd | THF |

### Example 8-15

This example demonstrates experiments to catalytically convert PAP to PPD. In a typical experiment, 0.1 g of catalyst, 1.0 g of PAP, and 30 mL of solvent are placed in 75 mL high pressure Parr reactor. The reactor is sealed and then pressurized, first, with NH₃. The reaction was then pressurized with H₂ for reactions using both gases. The reactor is heated up to the target temperature and the temperature is maintained for 0.5 hour. The reaction product is obtained after the temperature reached room temperature. Analysis of the products is conducted by HPLC and GC. The example surveys multiple catalysts and conditions and the results are shown in Table 3.

TABLE 3
Example 8 Experiments to Convert PAP to PPD

| Expt. | Sub- | | | Reactant | Temp | Pressure |
|---|---|---|---|---|---|---|
| # | strate | Catalyst | Solvent | Gas | (° C.) | (NH₃/H₂, psi) |
| 8 | PAP | RaNi | H₂O | NH₃ | 150 | 75/300 |
| | PAP | RaNi | H₂O | NH₃ | 150 | 75/500 |
| | PAP | RaNi | H₂O | NH₃ | 300 | 75/300 |
| | PAP | RaNi | H₂O | NH₃ | 300 | 75/500 |
| 9 | PAP | RaNi | H₂O | NH₃/H₂ | 150 | 75/300 |
| | PAP | RaNi | H₂O | NH₃/H₂ | 150 | 75/500 |
| | PAP | RaNi | H₂O | NH₃/H₂ | 300 | 75/300 |
| | PAP | RaNi | H₂O | NH₃/H₂ | 300 | 75/500 |
| 10 | PAP | Ru/Al₂O₃ | H₂O | NH₃ | 150 | 75/300 |
| | PAP | Ru/Al₂O₃ | H₂O | NH₃ | 150 | 75/500 |
| | PAP | Ru/Al₂O₃ | H₂O | NH₃ | 300 | 75/300 |
| | PAP | Ru/Al₂O₃ | H₂O | NH₃ | 300 | 75/500 |
| | PAP | Ru/Al₂O₃ | H₂O | NH₃/H₂ | 150 | 75/300 |
| | PAP | Ru/Al₂O₃ | H₂O | NH₃/H₂ | 150 | 75/500 |
| | PAP | Ru/Al₂O₃ | H₂O | NH₃/H₂ | 300 | 75/300 |
| | PAP | Ru/Al₂O₃ | H₂O | NH₃/H₂ | 300 | 75/500 |
| 11 | PAP | Ru/Al₂O₃ | THF | NH₃ | 150 | 75/300 |
| | PAP | Ru/Al₂O₃ | THF | NH₃ | 150 | 75/500 |
| | PAP | Ru/Al₂O₃ | THF | NH₃ | 300 | 75/300 |
| | PAP | Ru/Al₂O₃ | THF | NH₃ | 300 | 75/500 |
| | PAP | Ru/Al₂O₃ | THF | NH₃/H₂ | 150 | 75/300 |
| | PAP | Ru/Al₂O₃ | THF | NH₃/H₂ | 150 | 75/500 |
| | PAP | Ru/Al₂O₃ | THF | NH₃/H₂ | 300 | 75/300 |
| | PAP | Ru/Al₂O₃ | THF | NH₃/H₂ | 300 | 75/500 |
| 12 | PAP | Pd/C | H₂O | NH₃ | 150 | 75/300 |
| | PAP | Pd/C | H₂O | NH₃ | 150 | 75/500 |
| | PAP | Pd/C | H₂O | NH₃ | 300 | 75/300 |
| | PAP | Pd/C | H₂O | NH₃ | 300 | 75/500 |
| | PAP | Pd/C | H₂O | NH₃/H₂ | 150 | 75/300 |
| | PAP | Pd/C | H₂O | NH₃/H₂ | 150 | 75/500 |
| | PAP | Pd/C | H₂O | NH₃/H₂ | 300 | 75/300 |
| | PAP | Pd/C | H₂O | NH₃/H₂ | 300 | 75/500 |
| 13 | PAP | Pd/C | THF | NH₃ | 150 | 75/300 |
| | PAP | Pd/C | THF | NH₃ | 150 | 75/500 |
| | PAP | Pd/C | THF | NH₃ | 300 | 75/300 |
| | PAP | Pd/C | THF | NH₃ | 300 | 75/500 |
| | PAP | Pd/C | THF | NH₃/H₂ | 150 | 75/300 |
| | PAP | Pd/C | THF | NH₃/H₂ | 150 | 75/500 |
| | PAP | Pd/C | THF | NH₃/H₂ | 300 | 75/300 |
| | PAP | Pd/C | THF | NH₃/H₂ | 300 | 75/500 |

The same procedure is applied to demonstrate that N-(4-hydroxyphenyl)ethanamide can be converted to PPD. The results of these experiments are shown in Table 4.

TABLE 4
Example 8 Experiments to Convert N-(4-
hydroxyphenyl) ethanamide (NEA) to PPD

| Expt. | | | | Reactant | Temp | Pressure |
|---|---|---|---|---|---|---|
| # | Substrate | Catalyst | Solvent | Gas | (° C.) | (NH₃/H₂, psi) |
| 14 | NEA | Pd/C | H₂O | NH₃ | 150 | 75/300 |
| | NEA | Pd/C | H₂O | NH₃ | 150 | 75/500 |
| | NEA | Pd/C | H₂O | NH₃ | 300 | 75/300 |
| | NEA | Pd/C | H₂O | NH₃ | 300 | 75/500 |
| | NEA | Pd/C | H₂O | NH₃/H₂ | 150 | 75/300 |
| | NEA | Pd/C | H₂O | NH₃/H₂ | 150 | 75/500 |
| | NEA | Pd/C | H₂O | NH₃/H₂ | 300 | 75/300 |
| | NEA | Pd/C | H₂O | NH₃/H₂ | 300 | 75/500 |
| 15 | NEA | Pd/C | THF | NH₃ | 150 | 75/300 |
| | NEA | Pd/C | THF | NH₃ | 150 | 75/500 |
| | NEA | Pd/C | THF | NH₃ | 300 | 75/300 |
| | NEA | Pd/C | THF | NH₃ | 300 | 75/500 |
| | NEA | Pd/C | THF | NH₃/H₂ | 150 | 75/300 |
| | NEA | Pd/C | THF | NH₃/H₂ | 150 | 75/500 |
| | NEA | Pd/C | THF | NH₃/H₂ | 300 | 75/300 |
| | NEA | Pd/C | THF | NH₃/H₂ | 300 | 75/500 |

As described above, the preparation method of the present invention involves heterogeneous catalytic hydrogenolysis/decarboxylation and amination to efficiently produce highly pure PPD in the simple process. Major by-products are expected to be CO₂ and H₂O, without using and producing any halogenated compounds. The advantage of the method is simplified production with high selectivity, thereby requiring less effort on purification and isolation of product.

### Example 16

PABA (10 g) is added to 400 mL water in a 2 L round-bottom flask equipped with a mechanical stirrer and a condenser. Hydrochloric acid (100 mL 1.0 M) is added and the mixture is heated to 80° C. and maintained at that temperature overnight. The reaction is cooled, neutralized by addition of 2.0 M aqueous sodium hydroxide and most of the water (450 mL) is removed by distillation under vacuum. Aniline product separated from the aqueous salt solution as an oil, which can be separated physically or taken up in ethyl acetate and purified by distillation. The yield of crude aniline is 6.7 g, which is pure based on the proton NMR spectrum.

### Example 17

Solid PABA (50 g) and 100 mL diphenyl ether are added to a 500 mL round-bottom flask. The flask is heated to 200° C. with an oil bath and the temperature is maintained at that temperature until gas evolution ceases. The reaction is cooled and distilled under reduced pressure until aniline ceases to distill. The yield is 30 g, or 90% based on PABA.

### Example 18

Aniline prepared from biologically-derived PABA is condensed with formaldehyde in the following process. In a typical reaction, a 5 L reactor equipped with a condenser and mechanical stirrer was charged with 900 mL water, 1118 g aniline, 834 g hydrochloric acid (35% in water), and 324 g of formaldehyde solution (37% in water). The reactor was stirred and maintained at 30° C. during the charging process. After thorough mixing was completed, the reaction was heated to 90° C. and maintained at this temperature for 4 hours. The reaction was cooled and 800 g sodium hydroxide solution (50% in water) was added slowly. The reactor was heated to 95° C. with stirring to ensure thorough mixing and then allowed to cool to room temperature and sit for about one hour. The two-layer reaction mixture was separated and the upper layer (the “organic” layer) was heated under a slight vacuum to remove water and unreacted aniline, which was purified by distillation and recycled. Analysis by HPLC indicated that the crude product was 75% 4,4′-methylenedianiline, with the remainder being other methylenedianiline isomers and higher molecular weight condensation products of aniline and formaldehyde. The crude product can be distilled under vacuum to provide purified MDA that is essentially free of higher molecular weight aniline-formaldehyde condensation products.

The methylenedianiline prepared from so prepared aniline is identical in every respect to that prepared from petroleum derived aniline except for the higher ¹⁴C content of the MDA prepared from the aniline prepared from biologically-derived PABA.

### Example 19

In this example, PABA (either biologically-derived or petroleum-derived) is condensed directly with formaldehyde without prior conversion to aniline.

In a typical reaction, a 5 L reactor equipped with a condenser and mechanical stirrer was charged with 900 mL water, 1647 g PABA, 834 g hydrochloric acid (35% in water), and 324 g of formaldehyde solution (37% in water). The reactor was stirred and maintained at 30° C. during the charging process. After thorough mixing was completed, the reaction was heated to 90° C. and maintained at this temperature for 8 hours. The reaction was cooled and 800 g sodium hydroxide solution (50% in water) was added slowly. The reactor was heated to 95° C. with stirring to ensure thorough mixing and then allowed to cool to room temperature and sit for about one hour. The two-layer reaction mixture was separated and the upper layer (the “organic” layer) was heated under a slight vacuum to remove water and unreacted aniline, which was purified by distillation and recycled. Analysis by HPLC indicated that the crude product was 75% 4,4′-methylenedianiline, with the remainder being other methylenedianiline isomers and higher molecular weight condensation products of aniline and formaldehyde. The crude product can be distilled under vacuum to provide purified MDA that is essentially free of higher molecular weight aniline-formaldehyde condensation products.

The methylenedianiline prepared from petroleum-derived PABA or biologically-derived PABA are identical in every respect except for the higher ¹⁴C content of the MDA prepared from the biologically-derived PABA.

### Example 20

This example demonstrates the preparation of biologically-derived methylene diphenyl diisocyanate (MDI) from the crude bio-derived MDA prepared in Example 18 above. The MDA (122 g, 0.615 mol) was dissolved in 1.0 L of dry chlorobenzene and added to a chilled (10° C.) solution of phosgene (100 g, 1.01 moles) in chlorobenzene (400 mL) in a 5 L flask equipped with a mechanical stirrer and condenser. After the addition was complete, the reaction mixture was warmed to room temperature over 30 minutes and then slowly heated to reflux. An additional 375 g (3.79 moles) of phosgene in 1.0 L chlorobenzene was added over 5 hr while maintaining the reaction at reflux. The reaction was heated for an additional hour and then purged with nitrogen (the purged gas was passed through a trap containing chilled aqueous sodium hydroxide). The chlorobenzene was then removed by distillation, with the final solvent removal being carried out under mild vacuum. The resulting crude MDI was analyzed spectroscopically (¹H-NMR, IR) and showed complete reaction of all amine functionality. The crude MDA can be used directly in applications such as formulation of adhesives and preparation of polyurethane foams or fractionally distilled under vacuum to give purified MDI, leaving poly-MDI in the distillation pot. The purified MDI can be used in preparation of high performance polyurethane rubbers as elastomers.

The MDI prepared from biologically-derived MDA is identical in every respect to that prepared from petroleum-derived MDA except for the ¹⁴C content of the MDI prepared from the biologically-derived MDI.

### SEQUENCE LISTINGS

TABLE 5
Gene sequences and corresponding amino acid sequences for E. coli B Strain REL606

aroF^{FBR}
atgcaaaaagacgcgctgaataacgtacatattaccgacgaacaggttttaatgactccggaacaactgaaggcc
gcttttccattgagcctgcaacaagaagcccagattgctgactcgcgtaaaaccatttcagatattatcgccggg
cgcgatcctcgtctgctggtagtatgtggtccttgttccattcatgatccggaaactgctctggaatatgctcgt
cgatttaaagcccttgccgcagaggtcagcgatagcctctatctggtaatgcgcgtctattttgaaaaaccccgt
accactgtcggctggaaagggttaattaacgatccccatatggatggctcttttgatgtagaagccgggctgcag
atcgcgcgtaaattgctgcttgagctggtgaatatgggactgccactggcgacggaagcgttagatcTgaatagc
ccgcaatacctgggcgatctgtttagctggtcagcaattggtgctcgtacaacggaatcgcaaactcaccgtgaa
atggcctccgggctttccatgccggttggttttaaaaacggcaccgacggcagtctggcaacagcaattaacgct
atgcgcgccgccgcccagccgcaccgttttgttggcattaaccaggcagggcaggttgcgttgctacaaactcag
gggaatccggacggccatgtgatcctgcgcggtggtaaagcgccgaactatagccctgcggatgttgcgcaatgt
gaaaaagagatggaacaggcgggactgcgcccgtctctgatggtagattgcagccacggtaattccaataaagat
tatcgccgtcagcctgcggtggcagaatccgtggttgctcaaatcaaagatggcaatcgctcaattattggtctg
atgatcgaaagtaatatccacgagggcaatcagtcttccgagcaaccgcgcagtgaaatgaaatacggtgtatcc
gtaaccgatgcctgcattagctgggaaatgaccgatgccttgctgcgtgaaattcatcaggatctgaacgggcag
ctgacggctcgcgtggcttaa
AroF^{FBR}
MQKDALNNVHITDEQVLMTPEQLKAAFPLSLQQEAQIADSRKTISDIIAGRDPRLLVVCGPCSIHDPETALE
YARRFKALAAEVSDSLYLVMRVYFEKPRTTVGWKGLINDPHMDGSFDVEAGLQIARKLLLELVNMGLPLATE
ALDLNSPQYLGDLFSWSAIGARTTESQTHREMASGLSMPVGFKNGTDGSLATAINAMRAAAQPHRFVGINQA
GQVALLQTQGNPDGHVILRGGKAPNYSPADVAQCEKEMEQAGLRPSLMVDCSHGNSNKDYRRQPAVAESVVA
QIKDGNRSIIGLMIESNIHEGNQSSEQPRSEMKYGVSVTDACISWEMTDALLREIHQDLNGQLTARVA
aroB
atggagaggattgtcgttactctcggggaacgtagttacccaattaccatcgcatctggtttgtttaatgaacca
gcttcattcttaccgctgaaatcgggcgagcaggtcatgttggtcaccaacgaaaccctggctcctctgtatctc
gataaggtccgcggcgtacttgaacaggcgggtgttaacgtcgatagcgttatcctccctgacggcgagcagtat
aaaagcctggctgtactcgataccgtctttacggcgttgttacaaaagccgcatggtcgcgatactacgctggtg
gcgcttggcggcggcgtagtgggcgatctgaccggcttcgcggcggcgagttatcagcgcggtgttcgtttcatt
caagtcccgacgacgttactgtcgcaggtcgattcctccgttggcggcaaaactgcggtcaaccatcccctcggt
aaaaacatgattggcgcgttctaccagcctgcttcagtggtggtggatctcgactgtctgaaaacgcttcccccg
cgtgagttagcgtcggggctggcagaagtcatcaaatacggcattattcttgacggtgcgttttttaactggctg
gaagagaatctggatgcgttgttgcgtctggacggtccggcaatggcgtactgtattcgccgttgttgtgaactg
aaggcagaagttgtcgccgccgacgagcgcgaaaccgggttacgtgctttactgaatctgggacacacctttggt
catgccattgaagctgaaatggggtatggcaattggttacatggtgaagcggtcgctgcgggtatggtgatggcg
gcgcggacgtcggaacgtctcgggcagtttagttctgccgaaacgcagcgtattataaccctgctcaagcgggct
gggttaccggtcaatgggccgcgcgaaatgtccgcgcaggcgtatttaccgcatatgctgcgtgacaagaaagtc
cttgcgggagagatacgcttaattcttccgttggcaattggtaagagtgaagttcgcagcggcgtttcgcacgag
cttgttcttaacgccattgccgattgtcaatcagcgtaa
AroB
MERIVVTLGERSYPITIASGLFNEPASFLPLKSGEQVMLVTNETLAPLYLDKVRGVLEQAGVNVDSVILPDGEQ
YKSLAVLDTVFTALLQKPHGRDTTLVALGGGVVGDLTGFAAASYQRGVRFIQVPTTLLSQVDSSVGGKTAVNHP
LGKNMIGAFYQPASVVVDLDCLKTLPPRELASGLAEVIKYGIILDGAFFNWLEENLDALLRLDGPAMAYCIRRC
CELKAEVVAADERETGLRALLNLGHTFGHAIEAEMGYGNWLHGEAVAAGMVMAARTSERLGQFSSAETQRIITL
LKRAGLPVNGPREMSAQAYLPHMLRDKKVLAGEIRLILPLAIGKSEVRSGVSHELVLNAIADCQSA
aroD
atgaaaaccgtaactgtaaaagatctcgtcattggtgcgggcgcacctaaaatcatcgtctcgctgatggcgaaa
gatatcgcccgcgtgaaatccgaagctctcgcctatcgtgaagcggactttgatattctggaatggcgtgtggac
cactttgccgacctctccaatgtggagtctgtcatggcggcggcaaaaattctccgtgaaaccatgccagaaaaa
ccgctgctgtttaccttccgcagtgccaaagaaggcggcgagcaggcgatttccaccgaggcttatattgctctc
aatcgtgcagccatcgacagcggcctggttgatatgatcgatctggagttatttaccggcgatgatcaggttaaa
gaaaccgtcgcctacgcccacgcgcatgatgtgaaagttgtcatgtccaaccatgacttccataaaacgccggaa
gccgaagaaatcattgcccgtctgcgcaaaatgcaatccttcgacgccgatattcctaagattgcgctgatgccg
caaagtaccagcgatgtgctgacgttgcttgccgcgaccctggagatgcaggagcagtatgccgatcgtccaatc
atcacgatgtcgatggcaaaaactggcgtaatttctcgtctggctggtgaagtatttgggtcggcggcaactttt
ggtgcggtaaaaaaagcctctgcgccagggcaaatctcggtaactgatttacgcacagtattaactattttacat
caggcataa
AroD
MKTVTVKDLVIGAGAPKIIVSLMAKDIARVKSEALAYREADFDILEWRVDHFADLSNVESVMAAAKILRET
MPEKPLLFTFRSAKEGGEQAISTEAYIALNRAAIDSGLVDMIDLELFTGDDQVKETVAYAHAHDVKVVMSN
HDFHKTPEAEEIIARLRKMQSFDADIPKIALMPQSTSDVLTLLAATLEMQEQYADRPIITMSMAKTGVISR
LAGEVFGSAATFGAVKKASAPGQISVTDLRTVLTILHQA
aroE
atggaaacctatgctgtttttggtaatccgatagcccacagcaaatcgccattcattcatcagcaatttgctcagc
aactgaatattgaacatccctatgggcgcgtgttggcacccatcaatgatttcatcaacacactgaacgctttctt
tagtgctggtggtaaaggtgcgaatgtgacggtgccttttaaagaagaggcttttgccagagcggatgagcttact
gaacgggcagcgttggctggtgctgttaataccctcatgcggttagaagatggacgcctgctgggtgacaataccg
atggtgtaggcttgttaagcgatctggaacgtctgtcttttatccgccctggtttacgtattctgcttatcggcgc
tggtggagcatctcgcggcgtactactgccactcctttccctggactgtgcggtgacaataactaatcggacggta
tcccgcgcggaagagttggctaaattgtttgcgcacactggcagtattcaggcgttgagtatggacgaactggaag
gtcatgagtttgatctcattattaatgcaacatccagtggcatcagtggtgatattccggcgatcccgtcatcgct
cattcatccaggcatttattgctatgacatgttctatcagaaaggaaaaactccttttctggcatggtgtgagcag
cgaggctcaaagcgtaatgctgatggtttaggaatgctggtggcacaggcggctcatgcctttcttctctggcacg
gtgttctgcctgacgtagaaccagttataaagcaattgcaggaggaattgtccgcgtga
AroE
METYAVFGNPIAHSKSPFIHQQFAQQLNIEHPYGRVLAPINDFINTLNAFFSAGGKGANVTVPFKEEAFARADELT
ERAALAGAVNTLMRLEDGRLLGDNTDGVGLLSDLERLSFIRPGLRILLIGAGGASRGVLLPLLSLDCAVTITNRTV
SRAEELAKLFAHTGSIQALSMDELEGHEFDLIINATSSGISGDIPAIPSSLIHPGIYCYDMFYQKGKTPFLAWCEQ
RGSKRNADGLGMLVAQAAHAFLLWHGVLPDVEPVIKQLQEELSA
aroL
atgacacaacctctttttctgatcgggcctcggggctgtggtaaaacaacggtcggaatggcccttgccgattcgc
ttaaccgtcggtttgtcgataccgatcagtggttgcaatcacagctcaatatgacggtcgcggagatcgtcgaaag
ggaagagtgggcgggatttcgcgccagagaaacggcggcgctggaagcggtaactgcgccatccaccgttatcgct
acaggcggcggcattattctgacggaatttaatcgtcacttcatgcaaaataacgggatcgtggtttatttgtgtg
cgccagtatcagtcctggttaaccgactgcaagctgcaccggaagaagatttacggccaaccttaacgggaaaacc
gctgagcgaagaagttcaggaagtgctggaagaacgcgatgcgctatatcgcgaagttgcgcatattatcatcgac
gcaacaaacgaacccagccaggtgatttctgaaattcgtagcgccctggcacagacgatcaattgttga
AroL
MTQPLFLIGPRGCGKTTVGMALADSLNRRFVDTDQWLQSQLNMTVAEIVEREEWAGFRARETAALEAVTAPST
VIATGGGIILTEFNRHFMQNNGIVVYLCAPVSVLVNRLQAAPEEDLRPTLTGKPLSEEVQEVLEERDALYREV
AHIIIDATNEPSQVISEIRSALAQTINC
aroA
atggaatccctgacgttacaacccatcgctcgtgtcgatggcactattaatctgcccggttccaagagcgtttcta
accgcgctttattgctggcggcattagcacacggcaaaacagtattaaccaatctgctggatagcgatgacgtgcg
ccatatgctgaatgcattaacagggttaggggtaagctatacgctttcagccgatcgtacgcgttgcgaaattatc
ggtaacggcggtccattacacgcagaaggtgccctggagttgttccacggcaatgcgtccgctggcggcagctctt
tgtctgggtagcaatgatattgtgctgaccggtgagccgcgtatgaaagaacgcccgattggtcatctggtggatg
ctctgcgcctgggcggggcgaagatcacttacctggaacaagaaaattatccgccgttgcgtttacagggcggctt
taccggcggcaacgttgacgttgatggctccgtttccagccaattcctcaccgcactgttaatgactgcgcctctt
gcgccggaagatacggtgattcgtattaaaggcgatctggtttctaaaccttatatcgacatcacactcaatctga
tgaagacgttlggtgttgaaattgaaaatcagcactatcaacaatttgtcgtaaaaggcgggcagtcttatcagtc
tccgggtacttatttggtcgaaggcgatgcatcttcggcttcttactttctggcagcagcagcaatcaaaggcggc
actgtaaaagtgaccggtattggacgtaacagtatgcagggtgatattcgcrttgctgatgtgctggaaaaaatgg
gcgcgaccatttgctggggcgatgattatatttcctgcacgcgtggtgaactgaacgctattgatatggatatgaa
ccatattcccgatgcggcgatgaccattgccacggcggcgttatttgcaaaaggcaccaccacgctgcgcaatatc
tataactggcgtgttaaagaaaccgatcgcctgtttgcgatggcaacagaactgcgtaaagtcggtgcggaagtag
aagaggggcacgattacattcgtatcactccaccggaaaaactgaactttgccgagatcgcgacatacaatgatca
ccggatggcgatgtgtttctcgctggtggcgttgtcagatacaccagtgacgattcttgatcccaaatgcacggcc
aaaacatttccggattatttcgagcagctggcgcggattagccaggcagcctga
AroA
MESLTLQPIARVDGTINLPGSKSVSNRALLLAALAHGKTVLTNLLDSDDVRHMLNALTGLGVSYTLSADRTR
CEIIGNGGPLHAEGALELFLGNAGTAMRPLAAALCLGSNDIVLTGEPRMKERPIGHLVDAIRLGGAKITYLE
QENYPPLRLQGGFTGGNVDVDGSVSSQFLTALLMTAPLAPEDTVIRIKGDLVSKPYIDITLNLMKTFGVEIE
NQHYQQFVVKGGQSYQSPGTYLVEGDASSASYFLAAAAIKGGTVKVTGIGRNSMQGDIRFADVLEKMGATIC
WGDDYISCTRGELNAIDMDMNHIPDAAMTIATAALFAKGTTTLRNIYNWRVKETDRLFAMATELRKVGAEVE
EGHDYIRITPPEKLNFAEIATYNDHRMAMCFSLVALSDTPVTILDPKCTAKTFPDYFEQLARISQAA
aroC
atggctggaaacacaattggacaactctttcgcgtaaccactttcggcgaatcgcacgggctggcgctcggctgca
tcgtcgatggtgttccgccaggcattccgctgacggaagcggacctgcaacatgacctcgaccgtcgtcgccctgg
gacatcgcgctataccacccagcgccgcgagccggatcaggtcaaaattctctccggtgtttttgagggcgttacc
accggcaccagcattggcttgttgatcgaaaataccgaccagcgttctcaggattacagcgcaattaaagacgttt
tccgcccaggccatgctgattacacctacgaacaaaaatacggtctgcgcgattatcgcggcggtggacgttcttc
cgcccgcgaaaccgccatgcgcgtagcggcgggggcgattgccaaaaaatatctcgctgagaaatttggcatcgaa
attcgcggctgcctgacccagatgggcgacattccgctggaaatcaaagactggtcgcaggtcgagagttgatgcg
cgcgctgaaaaaagagggcgactccatcggcgcgaaagtcaccgttgttgccagtggcgtccccgccggacttggc
gagccggtctttgaccgcctggatgccgacatcgcccatgcgctaatgagcatcaacgcggtgaaaggcgtggaaa
ttggcgacggttttgacgtggtggcgctgcgcggcagccagaatcgcgacgaaatcaccaaagacggtttccagag
caaccatgcgggcggcattctcggcggtatcagcagcgggcagcaaatcattgcccatatggcgctgaaaccgacc
tccagcattaccgtgccgggtcgtaccattaaccgctttggcgaagaagttgagatgatcaccaaaggccgtcacg
atccctgtgtcgggatccgcgcagtgccgatcgcagaagcgatgctggcgatcgttttaatggatcacctgttacg
gcaacgggcgcaaaatgccgatgtgaagactgatattccacgctggtaa
AroC
MAGNTIGQLFRVTTFGESHGLALGCIVDGVPPGIPLTEADLQHDLDRRRPGTSRYTTQRREPDQVKILSGVFE
GVTTGTSIGLLIENTDQRSQDYSAIKDVFRPGHADYTYEQKYGLRDYRGGGRSSARETAMRVAAGAIAKKYLA
EKFGIEIRGCLTQMGDIPLEIKDWSQVEQNPFFCPDPDKIDALDELMRALKKEGDSIGAKVTVVASGVPAGLG
EPVFDRLDADIAHALMSINAVKGVEIGDGFDVVALRGSQNRDEITKDGFQSNHAGGILGGISSGQQIIAHMAL
KPTSSITVPGRTINRFGEEVEMITKGRHDPCVGIRAVPIAEAMLAIVLMDHLLRQRAQNADVKTDIPRW
pabA
atgatcctgcttatagataactacgattcttttacctggaacctctaccagtacttttgtgaactgggggcggatgtgctggttaagcgcaacga
tgcgttgacgctggcggatatcgacgcccttaaaccacaaaaaattgtcatctcacctggcccctgtacgccagatgaagccgggatctctcttg
acgttattcgccactatgccgggcgcttgccgattcttggcgtctgcctcggtcatcaggcaatggcgcaggcatttggcggtaaagttgtgcgc
aaaggtcatgcacggcaaaacctcgccgattacacataacggtgagggcgtatttcgggggctggcaaatccacttaccgtgacacgctaccatt
gccgccgctggtggtggaacctgactcattaccagcgtgctttgacgtgacggcctggagcgaaacccgcgagattatggggattcgccatcgcc
agtgggatctggaaggtgtgcagttccatccagaaagtattcttagcgaacaaggacatcaactgctggctaatttcctgcatcgctga
PabA
MILLIDNYDSFTWNLYQYFCELGADVLVKRNDALTLADIDALKPQKIVISPGPCTPDEAGISLDVIRHYAGRL
PILGVCLGHQAMAQAFGGKVVRAAKVMHGKTSPITHNGEGVFRGLANPLTVTRYHSLVVEPDSLPACFDVT
AWSETREIMGIRHRQWDLEGVQFHPESILSEQGHQLLANFLHR
pabB
atgaagacgttatctcccgctgtgattactttaccctggcgtcaggacgccgctgaattttatttctcccgcttaagccacctgccgtgggcga
tgcttttacactccggctatgccgatcatccgtatagccgctttgatattgtggtcgccgatccgatttgcactttaaccactttcggtaaaga
aaccgttgttagtgaaagcgaaaaacgcacaacgaccactgatgacccgctacaggtgctccagcaggtgctggatcgcgcagacattcgccca
acgcataacgaagatttgccatttcagggcggcgcactggggttgtttggctacgatctgggccgccgttttgagtcactgccagaaattgcgg
aacaagatatcgttctgccggatatggcagtgggtatctacgattgggcgctcattgtcgaccaccagcgtcatacagtttctttgctgagtca
taatgatgtcaatgcccgtcgggcctggctggaaagccagcaattctcgccgcaggaagatttcacgctcacttccgactggcaatccaatatg
acccgcgagcagtacggcgaaaaatttcgccaggtacaggaatatctgcacagcggtgattgctatcaggtgaatctcgcccagcgttttcatg
cgacctattctggcgatgaatggcaggcattccttcagcttaatcaggccaaccgcgcgccatttagcgcttttttacgtcttgaacagggtgc
aattttaagcctttcgccagagcggtttattctttgtgataatagtgaaatccagacccgcccgattaaaggcacgctaccacgcctgcccgat
cctcaggaagatagcaaacaagcagaaaaactggcgaactcagcgaaagatcgtgccgaaaatctgatgattgtcgatttaatgcgtaatgata
tcggtcgtgttgccgtagccggttcggtaaaagtaccagagctcttcgtggtggaacccttccctgccgtgcatcatctggtcagcactataac
ggcgcgactaccagaacagttacacgccagcgatctgctgcgcgcagcttttcctggtggctcaataaccggggctccgaaagtacgggctatg
gaaattatcgacgaactggaaccgcagcgacgtaatgcctggtgcggcagcattggctatttgagcttttgcggcaacatggataccagcatta
ctatccgcacgctgactgccattaacggacaaatatactgctctgcgggcggtggaattgtcgccgatagccaggaagaagcggaatatcagga
aacttttgataaagttaataagatattacgccaactggagaagtaa
PabB
MKTLSPAVITLPWRQDAAEFYFSRLSHLPWAMLLHSGYADHPYSRFDIVVADPICTLTTFGKETVVSESEKR
TTTTDDPLQVLQQVLDRADIRPTHNEDLPFQGGALGLFGYDLGRRFESLPEIAEQDIVLPDMAVGIYDWALI
VDHQRHTVSLLSHNDVNARRAWLESQQFSPQEDFTLTSDWQSNMTREQYGEKFRQVQEYLHSGDCYQVNLAQ
RFHATYSGDEWQAFLQLNQANRAPFSAFLRLEQGAILSLSPERFILCDNSEIQTRPIKGTLPRLPDPQEDSK
QAEKLANSAKDRAENLMIVDLMRNDIGRVAVAGSVKVPELFVVEPFPAVHHLVSTITARLPEQLHASDLLRA
AFPGGSITGAPKVRAMEIIDELEPQRRNAWCGSIGYLSFCGNMDTSITIRTLTAINGQIYCSAGGGIVADSQ
EEAEYQETFDKVNKILRQLEK
pabC
atgttcttaattaacggttataagcaggaatcgctggcagtaagcgatcgggcaacgcagtttggtgatggttgttttaccactgccagagt
tatcgacggtaaagtcagtttgttatcggcgcatatccagcgactacaggatgcttgtcagcggttgatgatttcctgtgacttctggcctc
agcttgaacaagagatgaaaacgctggcagcagaacagcaaaatggtgtactgaaagtcgtgatcagtcgcggtagtggcgggcgagggtac
agcacattgaacagcggaccagcaacgcggattctctccgttacggcttatcctgcacattacgaccgtttgcgtaacgaggggatgacgtt
gggtgctaagcccgtgcggctggggcgcaatcctcatcttgcaggtattaaacatcttaatcggcttgagcaagtattgattcgctctcatc
ttgagcagacaaacgctgatgaggcgctggtccttgacagcgaagggtgggttacggaatgctgtgcggctaatttgttctggcggaagggc
aacgtagtttatacgccgcgactggatcaggcaggtgttaacggcattatgcgacaattctgtatccgtttgctggcacaatcctcttatca
gcttgtcgaagtgcaagcttctctggaagaggcgttgcaggcagatgagatggttatttgtaatgcgttaatgccagtgatgcccgtacgtg
cctgtggcgatgtctccttttcgtcagcaacgttatatgaatatttagccccactttgtgagcgcccgaattag
PabC
MFLINGYKQESLAVSDRATQFGDGCFTTARVIDGKVSLLSAHIQRLQDACQRLMISCDFWPQLEQEMKTLAAE
QQNGVLKVVISRGSGGRGYSTLNSGPATRILSVTAYPAHYDRLRNEGMTLVLSPVRLGRNPHLAGIKHLNRLE
QVLIRSHLEQTNADEALVLDSEGWVTECCAANLFWRKGNVVYTPRLDQAGVNGIMRQFCIRLLAQSSYQLVEV
QASLEEALQADEMVICNALMPVMPVRACGDVSFSSATLYEYLAPLCERPN
folP
atgaaactctttgcccagggtacttcactggaccttagccatcctcacgtaatggggatcctcaacgtcacgcctgattccttttcggatggtggc
acgcataactcgctgatagatgcggtgaaacatgcgaatctgatgatcaatgctggcgcgacgatcattgacgttggtggcgagtccacgcgccca
ggggcggcggaagttagcgttgaagaagagttgcaacgtgttattcctgtggttgaggcaattgctcaacgcttcgaagtctggatctcggtcgat
acatccaaaccagaagtcatccgtgagtcagcgaaagttggcgctcacattattaatgatatccgctccctttccgaacctggcgctctggaggcg
gctgcagaaaccggtttaccggtttgtctgatgcatatgcagggaaatccaaaaaccatgcaggaagctccgaagtatgacgatgtctttgcagaa
gtgaatcgctactttattgagcaaatagcacgttgcgagcaggcgggtatcgcaaaagagaaattgttgctcgaccccggattcggtttcggtaaa
aatctctcccataactattcattactggcgcgcctggctgaatttcaccatttcaacctgccgctgttggtgggtatgtcacgaaaatcgatgatt
gggcagctgctgaacgtggggccgtccgagcgcctgagcggtagtctggcctgtgcggtcattgccgcaatgcaaggcgcgcacatcattcgtgtt
catgacgtcaaagaaaccgtagaagcgatgcgggtggtggaagccactctgtctgcaaaggaaaacaaacgctatgagtaa
FolP
MKLFAQGTSLDLSHPHVMGILNVTPDSFSDGGTHNSLIDAVKHANLMINAGATIIDVGGESTRPGAAEVSVEE
ELQRVIPVVEAIAQRFEVVVISVDTSKPEVIRESAKVGAHIINDIRSLSEPGALEAAAETGLPVCLMHMQGNP
KTMQEAPKYDDVFAEVNRYFIEQIARCEQAGIAKEKLLLDPGFGFGKNLSHNYSLLARLAEFHHFNLPLLVGM
SRKSMIGQLLNVGPSERLSGSLACAVIAAMQGAHIIRVHDVKETVEAMRVVEATLSAKENKRYE
trpE
atgcaaacacaaaaaccgactctcgaacagctaacctgcgaaggcgcttatcgcgacaatcccaccgcgctttttcaccagttgtgtggggatc
gtccggcaacgctgctgctggaatccgcagatatcgacagcaaagatgatttaaaaagcctgctgctggtagacagtgcgctgcgcattacagc
tttaggtgacactgtcacaatccaggcactttccggcaacggcgaagccctgctggcactactggataacgccctgcctgcgggtgtggaaagt
gaacaatcaccaaactgccgtgtgctgcgcttcccccctgtcagtccactgctggatgaagacgcccgcttatgctccctttcggtttttgacg
ctttccgtttattgcagaatctgttgaatgtaccgaaggaagaacgagaagccatgttcttcggcggcctgttctcttatgaccttgtggcggg
atttgaagatttaccgcaactgtcagcggaaaataactgccctgatttctgtttttatctcgctgaaacgctgatggtgattgaccatcagaaa
aaaagcacccgtattcaggccagcctgtttgctccgaatgaagaagaaaaacaacgtctcactgctcgcctgaacgaactacgtcagcaactga
ccgaagccgcgccgccgctgccagtggtttccgtgccgcatatgcgttgtgaatgtaatcagagcgatgaagagttcggtggcgtagtgcgttt
gttgcaaaaagcgattcgcgctggagaaattttccaggtggtgccatctcgccgtttctctctgccctgcccgtcaccgctggcggcctattac
gtgctgaaaaagagtaatcccagcccgtacatgttttttatgcaggataatgatttcaccctatttggcgcgtcgccggaaagctcgctcaagt
atgatgccaccagccgccagattgagatctacccgattgccggaacacgcccacgcggtcgtcgcgccgatggttcactggacagagatctcga
cagccgtattgaactggaaatgcgtaccgatcataaagagctgtctgaacatctgatgctggttgatctcgcccgtaatgatctggcacgcatt
tgcacccccggcagccgctacgtcgccgatctcaccaaagttgaccgttattcctatgtgatgcacctcgtctctcgcgtagtcggcgaactgc
gtcacgatcttgacgccctgcacgcttatcgcgcctgtatgaatatggggacgttaagcggtgcgccgaaagtacgcgctatgcagttaattgc
cgaggcggaaggtcgtcgccgcggcagctacggcggcgcggtaggttatttcaccgcgcatggcgatctcgacacctgcattgtgatccgctcg
gcgctggtggaaaacggtatcgccaccgtgcaagcgggtgctggtgtagtccttgattctgttccgcagtcggaagccgacgaaacccgtaaca
aagcccgcgctgtactgcgcgctattgccaccgcgcatcatgcacaggagactttctga
TrpE
MQTQKPTLEQLTCEGAYRDNPTALFHQLCGDRPATLLLESADIDSKDDLKSLLLVDSALRITALGDTVTIQA
LSGNGEALLALLDNALPAGVESEQSPNCRVLRFPPVSPLLDEDARLCSLSVFDAFRLLQNLLNVPKEEREAM
FFGGLFSYDLVAGFEDLPQLSAENNCPDFCFYLAETLMVIDHQKKSTRIQASLFAPNEEEKQRLTARLNELR
QQLTEAAPPLPVVSVPHMRCECNQSDEEFGGVVRLLQKAIRAGEIFQVVPSRRFSLPCPSPLAAYYVLKKSN
PSPYMFFMQDNDFTLFGASPESSLKYDATSRQIEIYPIAGTRPRGRRADGSLDRDLDSRIELEMRTDHKELS
EHLMLVDLARNDLARICTPGSRYVADLTKVDRYSYVMHLVSRVVGELRHDLDALHAYRACMNMGTLSGAPKV
RAMQLIAEAEGRRRGSYGGAVGYFTAHGDLDTCIVIRSALVENGIATVQAGAGVVLDSVPQSEADETRNKAR
AVLRAIATAHHAQETF
trpD
atggctgacattctgctgctcgataatatcgactcttttacgtacaacctggcagatcagttgcgcagcaatggtcataacgtggtgatttaccg
caaccatattccggcgcagaccttaattgaacgcctggcgacgatgagcaatccggtactgatgctttctcctggccccggtgtgccgagcgaag
ccggttgtatgccggaactcctcacccgcttgcgtggcaagctgcccattattggcatttgcctcggacatcaggcaattgtcgaagcttacggg
ggctatgtcggtcaggcgggcgaaattcttcacggtaaagcgtcgagcattgaacatgacggtcaggcgatgtttgccggattaacaaacccgct
gccggtggcgcgttatcactcgctggttggcagtaacattccggccggtttaaccatcaacgcccattttaatggcatggtgatggcagtacgtc
acgatgcggatcgcgtttgtggattccagttccatccggaatccattctcaccacccagggcgctcgcctgctggaacaaacgctggcctgggcg
cagcagaaactagagccagccaacacgctgcaaccgattctggaaaaactgtatcaggcgcagacgcttagccaacaagaaagccaccagctgtt
ttcagcggtggtgcgtggcgagctgaagccggaacaactggcggcggcgctggtgagcatgaaaattcgcggtgagcacccgaacgagatcgccg
gagcagcaaccgcgctactggaaaacgccgcgccgttcccgcgcccggattatctgtttgctgatatcgtcggtactggcggtgacggcagcaac
agtatcaatatttctaccgccagtgcgtttgtcgccgcggcctgtgggctgaaagtggcgaaacacggcaaccgtagcgtctccagtaaatctgg
ttcgtccgatctgctggcggcgttcggtattaatcttgatatgaacgccgataaatcgcgccaggcgctggatgagttaggtgtatgtttcctct
ttgcgccgaagtatcacaccggattccgccacgcgatgccggttcgccagcaactgaaaacccgcaccctgttcaatgtgctggggccattgatt
aacccggcgcatccgccgctggcgttaattggtgtttatagtccggaactggtgctgccgattgccgaaaccttgcgcgtgctggggtatcaacg
cgcggcggtggtgcacagcggcgggatggatgaagtttcattacacgcgccgacaatcgttgccgagctgcatgacggcgaaattaagagctatc
aattgaccgctgaagattttggcctgactccctaccaccaggagcaactggcaggcggaacaccggaagaaaaccgtgacattttaacacgcttg
ttacaaggtaaaggcgacgccgcccatgaagcagccgtcgctgcgaacgtcgccatgttaatgcgcctgcatggccatgaagatctgcaagccaa
tgcgcaaaccgttcttgaggtactgcgcagtggttccgcttacgacagagttaccgcactggcggcacgagggtaa
TrpD
MADILLLDNIDSFTYNLADQLRSNGHNVVIYRNHIPAQTLIERLATMSNPVLMLSPGPGVPSEAGCMPELLTR
LRGKLPIIGICLGHQAIVEAYGGYVGQAGEILHGKASSIEHDGQAMFAGLTNPLPVARYHSLVGSNIPAGLTI
NAHFNGMVMAVRHDADRVCGFQFHPESILTTQGARLLEQTLAWAQQKLEPANTLQPILEKLYQAQTLSQQESH
QLFSAVVRGELKPEQLAAALVSMKIRGEHPNEIAGAATALLENAAPFPRPDYLFADIVGTGGDGSNSINISTA
SAFVAAACGLKVAKHGNRSVSSKSGSSDLLAAFGINLDMNADKSRQALDELGVCFLFAPKYHTGFRHAMPVRQ
QLKTRTLFNVLGPLINPAHPPLALIGVYSPELVLPIAETLRVLGYQRAAVVHSGGMDEVSLHAPTIVAELHDG
EIKSYQLTAEDFGLTPYHQEQLAGGTPEENRDILTRLLQGKGDAAHEAAVAANVAMLMRLHGHEDLQANAQTV
LEVLRSGSAYDRVTALAARG
pheA
atgacatcggaaaacccgttactggcgctgcgagagaaaatcagcgcgctggatgaaaaattattagcattactcgcagagcggcgcgaactgg
ccgtcgaggtgggaaaagccaaactgctctcgcatcgcccggtacgtgatattgatcgtgaacgcgatttactggaaagattaattacgctcgg
taaagcgcaccatctggacgcccattacattactcgcctgttccagctcatcattgaagattccgtattaactcagcaggctttgctccaacaa
catctcaataaaattaatccgcactcagcacgcatcgcttttctcggccccaaaggctcctattcacatcttgccgctcgtcagtacgctgccc
gtcactttgagcaattcattgaaagtggctgcgccaaatttgccgatatttttaatcaggtggaaaccggccaggccgactatgccgtcgtacc
gattgaaaataccagctccggtgccataaacgacgtttacgatctgctgcaacataccagcttgtcgattgttggcgagatgacgttaactatc
gaccattgtttgttggtctccggcactactgatttatccaccatcaatacggtctacagccatccgcagccattccagcaatgcagcaaattcc
ttaatcgttatccgcactggaagattgaatataccgaaagtacgtctgcggcaatggaaaaggttgcacaggcaaaatcaccgcatgttgctgc
gttgggaagcgaagctggcggcactttgtacggtttgcaggtactggagcgtattgaagcaaatcagcgacaaaacttcacccgatttgtggtg
ttggcgcgtaaagccattaacgtgtctgatcaggttccggcgaaaaccacgttgttaatggcgaccgggcaacaagccggtgcgctggttgaag
cgttgctggtactgcgcaaccacaatctgattatgacccgtctggaatcacgcccgattcacggtaatccatgggaagagatgttctatctgga
tattcaggccaatcttgaatcagcggaaatgcaaaaagcattgaaagagttaggggaaattacccgttcaatgaaggtattgggctgttaccca
agtgagaacgtagtgcctgttgatccaacctga
PheA
MTSENPLLALREKISALDEKLLALLAERRELAVEVGKAKLLSHRPVRDIDRERDLLERLITLGKAHHLDAHY
ITRLFQLIIEDSVLTQQALLQQHLNKINPHSARIAFLGPKGSYSHLAARQYAARHFEQFIESGCAKFADIFN
EQVTGQADYAVVPIENTSSGAINDVYDLLQHTSLSIVGEMTLTIDHCLLVSGTTDLSTINTVYSHPQPFQQC
SKFLNRYPHWKIEYTESTSAAMEKVAQAKSPHVAALGSEAGGTLYGLQVLERIEANQRQNFTRFVVLARKAI
NVSDQVPAKTTLLMATGQQAGALVEALLVLRNHNLIMTRLESRPIHGNPWEEMFYLDIQANLESAEMQKALK
ELGEITRSMKVLGCYPSENVVPVDPT
tyrA
atggttgctgaattgaccgcattacgcgatcaaattgatgaagtcgataaagcgctgctgaatttattagcgaagcgtctggaactggttgctga
agtgggcgaggtgaaaagccgctttggactgcctatttatgttccggagcgcgaggcatctatgttggcctcgcggcgcgcagaggcggaagctc
tgggtgtaccgccagatctgattgaggatgttttgcgtcgggtgatgcgtgaatcttactccagtgaaaacgacaaaggatttaaaacactttgt
ccgtcactgcgtccggtggttatcgtcggcggtggcggtcagatgggacgcctgttcgagaagatgctgacactatcgggttatcaggtgcggat
tctggagcaacatgactgggatcgagcggctgatattgttgccgatgccggaatggtgattgttagtgtgccaatccacgttactgagcaagtta
tcggcaaattaccgcctttaccgaaagattgtattctggttgatctggcatcagtgaaaaatggaccattacaggccatgctggcggcgcacgat
ggcccggtactggggttacacccgatgttcggcccggacagcggtagcctggcaaagcaagttgtggtctggtgtgatggacgtaagccggaagc
ataccaatggtttctggagcaaattcaggtctggggcgctcggctgcatcgtattagcgctgtcgagcacgatcagaatatggcgtttattcagg
ctctgcgccactttgctacttttgcttatgggctgcatctggcagaagaaaatgttcagcttgagcaacttctggcgctctcttcgccgatttac
cgccttgagctggcgatggtcgggcgactgtttgctcaggatccgcagctttatgccgacattattatgtcgtcagagcgtaatctggcgttaat
caaacgttactataagcgtttcggcgaggcgattgagttgctggagcagggcgataagcaggcgtttattgacagtttccgcaaggtggagcact
ggttcggcgattacgcacagcgttttcagagtgaaagccgcgtgttattgcgtcaggcgaatgacaaccgccagtaa
TyrA
MVAELTALRDQIDEVDKALLNLLAKRLELVAEVGEVKSRFGLPIYVPEREASMLASRRAEAEALGVPPDLI
EDVLRRVMRESYSSENDKGFKTLCPSLRPVVIVGGGGQMGRLFEKMLTLSGYQVRILEQHDWDRAADIVAD
AGMVIVSVPIHVTEQVIGKLPPLPKDCILVDLASVKNGPLQAMLAAHDGPVLGLHPMFGPDSGSLAKQVVV
WCDGRKPEAYQWFLEQIQVINGARLHRISAVEHDQNMAFIQALRHFATFAYGLHLAEENVQLEQLLALSSP
IYRLELAMVGRLFAQDPQLYADIIMSSERNLALIKRYYKRFGEAIELLEQGDKQAFIDSFRKVEHWFGDYA
QRFQSESRVLLRQANDNRQ
ubiC
atgtcacaccccgcgttaacgcaactgcgtgcgctgcgctattgtaaagagatccctgccctggatccgcaactgctcgactggctgttgctg
gaggattccatgacaaaacgttttgaacagcagggaaaaacggtaagcgtgacgatgatccgcgaagggtttgtcgagcagaatgaaatcccc
gaagaactgccgctgctgccgaaagagtctcgttactggttacgtgaaattttgttatgtgccgatggtgaaccgtggcttgccggtcgtacc
gtcgttcctgtgtcaacgttaagcgggccggagctggcgttacaaaaattgggtaaaacgccgttaggacgctatctgttcacatcatcgaca
ttaacccgggactttattgagataggccgtgatgccgggctgtgggggcgacgttcccgcctgcgattaagcggtaaaccgctgttgctaaca
gaactgtttttaccggcgtcaccgttgtactaa
UbiC
MSHPALTQLRALRYCKEIPALDPQLLDWLLLEDSMTKRFEQQGKTVSVTMIREGFVEQNEIPEELPLLPKES
RYWLREILLCADGEPWLAGRTVVPVSTLSGPELALQKLGKTPLGRYLFTSSTLTRDFIEIGRDAGLWGRRSR
LRLSGKPLLLTELFLPASPLY
entC
atggatacgtcactggctgaggaagtacagcagaccatggcaacacttgcgcccaatcgctttttctttatgtcgccgtaccgcagttttacgacg
tcaggatgtttcgcccgcttcgatgaaccggctgtgaacggggattcgcccgacagtcccttccagcaaaaactcgccgcgctgtttgccgatgcc
aaagcgcagagcatcaaaaatccggtgatagtcggggcgattcccttcgatccacgtcagccttcgtcgctgtatattcccgaatcctggcagtcg
ttctcccgccaggaaaaacagacctcagcccgccgttttacccgcagccagtcgctgaacgtggtggaacgccaggcaattcctgaacaaaccacg
tttgaacagatggttgctcgcgctgccgaacttaccgccacgccgcaggtcgacaaagtggtgttgtcacggttgattgatatcaccactgacgcc
gccattgatagtggcgtattgctggaacggttgattgcgcaaaacccggttagttacaacttccatgtcccgctggctgatggtggcgtcctgctg
ggggccagcccggaactgctgctacgtaaagacggcgagcgtttagctccattccgttagccggttccgcgcgtcgtcagccggatgaagtctcga
tcgcgaagcgggtaatcgtctgctggcgtcagaaaaagatcgccatgaacatgaactggtgactcaggcgatgaaagaggtactgcgcgaacgcag
tagtgagttacacgttccctcctctccacaattgattaccacgccgacgctgtggcatctcgcaactccctttgaaggtaaagcgaattcgcaaga
aaacgcactgactctggcctgtctgctgcatccaacccccgcgctgagcggtttcccgcatcaggccgcgacccaggttattgctgaactggagcc
attcgaccgcgaactgtttggcggcattgtgggttggtgtgacagcgaaggtaacggcgaatgggtggtgaccatccgctgcgcgaagctgcggga
aaatcaggtgcgtctgtttgccggagcggggattgtgcctgcgtcgtcaccgttgggtgagtggcgcgaaacaggcgtcaaactttctaccatgtt
gaacgtttttggattgcattaa
EntC
MDTSLAEEVQQTMATLAPNRFFFMSPYRSFTTSGCFARFDEPAVNGDSPDSPFQQKLAALFADAKAQSIKN
PVIVGAIPFDPRQPSSLYIPESWQSFSRQEKQTSARRFTRSQSLNVVERQAIPEQTTFEQMVARAAELTAT
PQVDKVVLSRLIDITTDAAIDSGVLLERLIAQNPVSYNFHVPLADGGVLLGASPELLLRKDGERFSSIPLA
GSARRQPDEVLDREAGNRLLASEKDRHEHELVTQAMKEVLRERSSELHVPSSPQLITTPTLWHLATPFEGK
ANSQENALTLACLLHPTPALSGFPHQAATQVIAELEPFDRELFGGIVGWCDSEGNGEWVVTIRCAKLRENQ
VRLFAGAGIVPASSPLGEWRETGVKLSTMLNVFGLH
menF
gtgcaatcacttactacggcgctggaaaatctactgcgccatttgtcgcaagagattccggcgacacccggcattcgggttatcgatattccttt
ccctctcaaagacgcttttgatgccttgagctggctggccagtcagcaaacatacccgcaattctactggcaacaacgtaatggtgatgaagaag
ctgtcgtcctgggcgcgattacccgttttacgtcgttggaccaggcacaacgttttcttcgccagcacccggaacacgccgacttacgcatttgg
gggctgaatgcttttgacccgtcgcagggcaatttacttttaccccgcctggaatggcgacgctgtggcggtaaagccacgctgcggctgacgct
attcagcgaaagctcccttcagcacgatgcgattcaggcaaaagaatttatcgccacactggtgagtatcaagcccttgcctgggttacatttaa
ccaccacgcgagaacaacactggccggacaaaacgggctggacgcaattaatcgaactggcaacgaaaaccatcgccgaaggtgagctcgacaaa
gtggtgctcgctcgggcaactgacctgcatttcgcaagtccggtcaacgcggcggcgatgatggctgccagtcgtcgactgaatctgaattgcta
ccatttttacatggcctttgatggcgaaaatgcttttcttggctcttcaccggaacggttatggcggcggcgtgacaaagcgctgcgtactgaag
cgctggcgggaacagtagcaaataatcctgatgataagcaggcgcagcagttaggagagtggctgatggcggatgataaaaaccagcgcgagaac
atgctggtggtggaagatatctgtcaacgattacaggccgatacccagacgctggatgttttaccgccgcaggtactgcgtctgcgtaaagtgca
gcatcttcgccgctgtatctggacttcactcaacaaagcggatgatgtgatctgtttacatcagttgcagccaacggcagcagttgctggcttac
cgcgcgatctggcgcgacagtttatcgcccgtcacgaaccgttcacccgagaatggtacgccggttctgcgggctatctctcattacaacaaagc
gaattctgcgtttccctgcgctcagcaaaaattagcggcaatgtcgtgcgattatatgctggcgcgggcattgtccgtggttccgaccccgagca
agagtggcaggaaatcgacaacaaagcggcagggctgcgtactttattacaaatggaatag
MenF
VQSLTTALENLLRHLSQEIPATPGIRVIDIPFPLKDAFDALSWLASQQTYPQFYWQQRNGDEEAVVLGAITR
FTSLDQAQRFLRQHPEHADLRIWGLNAFDPSQGNLLLPRLEWRRCGGKATLRLTLFSESSLQHDAIQAKEFI
ATLVSIKPLPGLHLTTTREQHWPDKTGWTQLIELATKTIAEGELDKVVLARATDLHFASPVNAAAMMAASRR
LNLNCYHFYMAFDGENAFLGSSPERLWRRRDKALRTEALAGTVANNPDDKQAQQLGEWLMADDKNQRENMLV
VEDICQRLQADTQTLDVLPPQVLRLRKVQHLRRCIWTSLNKADDVICLHQLQPTAAVAGLPRDLARQFIARH
EPFTREWYAGSAGYLSLQQSEFCVSLRSAKISGNVVRLYAGAGIVRGSDPEQEWQEIDNKAAGLRTLLQME

TABLE 6
Gene sequences and corresponding amino acid sequences for
Saccharomyces cerevisiae

ARO4
ATGAGTGAATCTCCAATGTTCGCTGCCAACGGCATGCCAAAGGTAAATCAAGGTGCTGAAGAAGATGTCA
GAATTTTAGGTTACGACCCATTAGCTTCTCCAGCTCTCCTTCAAGTGCAAATCCCAGCCACACCAACTTC
TTTGGAAACTGCCAAGAGAGGTAGAAGAGAAGCTATAGATATTATTACCGGTAAAGACGACAGAGTTCTT
GTCATTGTCGGTCCTTGTTCCATCCATGATCTAGAAGCCGCTCAAGAATACGCTTTGAGATTAAAGAAAT
TGTCAGATGAATTAAAAGGTGATTTATCCATCATTATGAGAGCATACTTGGAGAAGCCAAGAACAACCGT
CGGCTGGAAAGGTCTAATTAATGACCCTGATGTTAACAACACTTTCAACATCAACAAGGGTTTGCAATCC
GCTAGACAATTGTTTGTCAACTTGACAAATATCGGTTTGCCAATTGGTTCTGAAATGCTTGATACCATTT
CTCCTCAATACTTGGCTGATTTGGTCTCCTTCGGTGCCATTGGTGCCAGAACCACCGAATCTCAACTGCA
CAGAGAATTGGCCTCCGGTTTGTCTTTCCCAGTTGGTTTCAAGAACGGTACCGATGGTACCTTAAATGTT
GCTGTGGATGCTTGTCAAGCCGCTGCTCATTCTCACCATTTCATGGGTGTTACTAAGCATGGTGTTGCTG
CTATCACCACTACTAAGGGTAACGAACACTGCTTCGTTATTCTAAGAGGTGGTAAAAAGGGTACCAACTA
CGACGCTAAGTCCGTTGCAGAAGCTAAGGCTCAATTGCCTGCCGGTTCCAACGGTCTAATGATTGACTAC
TCTCACGGTAACTCCAATAAGGATTTCAGAAACCAACCAAAGGTCAATGACGTTGTTTGTGAGCAAATCG
CTAACGGTGAAAACGCCATTACCGGTGTCATGATTGAATCAAACATCAACGAAGGTAACCAAGGCATCCC
AGCCGAAGGTAAAGCCGGCTTGAAATATGGTGTTTCCATCACTGATGCTTGTATAGGTTGGGAAACTACT
GAAGACGTCTTGAGGAAATTGGCTGCTGCTGTCAGACAAAGAAGAGAAGTTAACAAGAAATAG
Aro4p
MSESPMFAANGMPKVNQGAEEDVRILGYDPLASPALLQVQIPATPTSLETAKRGRREAIDIITGKDDRVLVI
VGPCSIHDLEAAQEYALRLKKLSDELKGDLSIIMRAYLEKPRTTVGWKGLINDPDVNNTFNINKGLQSARQL
FVNLTNIGLPIGSEMLDTISPQYLADLVSFGAIGARTTESQLHRELASGLSFPVGFKNGTDGTLNVAVDACQ
AAAHSHHFMGVTKHGVAAITTTKGNEHCFVILRGGKKGTNYDAKSVAEAKAQLPAGSNGLMIDYSHGNSNKD
FRNQPKVNDVVCEQIANGENAITGVMIESNINEGNQGIPAEGKAGLKYGVSITDACIGWETTEDVLRKLAAA
VRQRREVNKK
ARO1
ATGGTGCAGTTAGCCAAAGTCCCAATTCTAGGAAATGATATTATCCACGTTGGGTATAACATTCATGACC
ATTTGGTTGAAACCATAATTAAACATTGTCCTTCTTCGACATACGTTATTTGCAATGATACGAACTTGAG
TAAAGTTCCATACTACCAGCAATTAGTCCTGGAATTCAAGGCTTCTTTGCCAGAAGGCTCTCGTTTACTT
ACTTATGTTGTTAAACCAGGTGAGACAAGTAAAAGTAGAGAAACCAAAGCGCAGCTAGAAGATTATCTTT
TAGTGGAAGGATGTACTCGTGATACGGTTATGGTAGCGATCGGTGGTGGTGTTATTGGTGACATGATTGG
GTTCGTTGCATCTACATTTATGAGAGGTGTTCGTGTTGTCCAAGTACCAACATCCTTATTGGCAATGGTC
GATTCCTCCATTGGTGGTAAAACTGCTATTGACACTCCTCTAGGTAAAAACTTTATTGGTGCATTTTGGC
AACCAAAATTTGTCCTTGTAGATATTAAATGGCTAGAAACGTTAGCCAAGAGAGAGTTTATCAATGGGAT
GGCAGAAGTTATCAAGACTGCTTGTATTTGGAACGCTGACGAATTTACTAGATTAGAATCAAACGCTTCG
TTGTTCTTAAATGTTGTTAATGGGGCAAAAAATGTCAAGGTTACCAATCAATTGACAAACGAGATTGACG
AGATATCGAATACAGATATTGAAGCTATGTTGGATCATACATATAAGTTAGTTCTTGAGAGTATTAAGGT
CAAAGCGGAAGTTGTCTCTTCGGATGAACGTGAATCCAGTCTAAGAAACCTTTTGAACTTCGGACATTCT
ATTGGTCATGCTTATGAAGCTATACTAACCCCACAAGCATTACATGGTGAATGTGTGTCCATTGGTATGG
TTAAAGAGGCGGAATTATCCCGTTATTTCGGTATTCTCTCCCCTACCCAAGTTGCACGTCTATCCAAGAT
TTTGGTTGCCTACGGGTTGCCTGTTTCGCCTGATGAGAAATGGTTTAAAGAGCTAACCTTACATAAGAAA
ACACCATTGGATATCTTATTGAAGAAAATGAGTATTGACAAGAAAAACGAGGGTTCCAAAAAGAAGGTGG
TCATTTTAGAAAGTATTGGTAAGTGCTATGGTGACTCCGCTCAATTTGTTAGCGATGAAGACCTGAGATT
TATTCTAACAGATGAAACCCTCGTTTACCCCTTCAAGGACATCCCTGCTGATCAACAGAAAGTTGTTATC
CCCCCTGGTTCTAAGTCCATCTCCAATCGTGCTTTAATTCTTGCTGCCCTCGGTGAAGGTCAATGTAAAA
TCAAGAACTTATTACATTCTGATGATACTAAACATATGTTAACCGCTGTTCATGAATTGAAAGGTGCTAC
GATATCATGGGAAGATAATGGTGAGACGGTAGTGGTGGAAGGACATGGTGGTTCCACATTGTCAGCTTGT
GCTGACCCCTTATATCTAGGTAATGCAGGTACTGCATCTAGATTTTTGACTTCCTTGGCTGCCTTGGTCA
ATTCTACTTCAAGCCAAAAGTATATCGTTTTAACTGGTAACGCAAGAATGCAACAAAGACCAATTGCTCC
TTTGGTCGATTCTTTGCGTGCTAATGGTACTAAAATTGAGTACTTGAATAATGAAGGTTCCCTGCCAATC
AAAGTTTATACTGATTCGGTATTCAAAGGTGGTAGAATTGAATTAGCTGCTACAGTTTCTTCTCAGTACG
TATCCTCTATCTTGATGTGTGCCCCATACGCTGAAGAACCTGTAACTTTGGCTCTTGTTGGTGGTAAGCC
AATCTCTAAATTGTACGTCGATATGACAATAAAAATGATGGAAAAATTCGGTATCAATGTTGAAACTTCT
ACTACAGAACCTTACACTTATTATATTCCAAAGGGACATTATATTAACCCATCAGAATACGTCATTGAAA
GTGATGCCTCAAGTGCTACATACCCATTGGCCTTCGCCGCAATGACTGGTACTACCGTAACGGTTCCAAA
CATTGGTTTTGAGTCGTTACAAGGTGATGCCAGATTTGCAAGAGATGTCTTGAAACCTATGGGTTGTAAA
ATAACTCAAACGGCAACTTCAACTACTGTTTCGGGTCCTCCTGTAGGTACTTTAAAGCCATTAAAACATG
TTGATATGGAGCCAATGACTGATGCGTTCTTAACTGCATGTGTTGTTGCCGCTATTTCGCACGACAGTGA
TCCAAATTCTGCAAATACAACCACCATTGAAGGTATTGCAAACCAGCGTGTCAAAGAGTGTAACAGAATT
TTGGCCATGGCTACAGAGCTCGCCAAATTTGGCGTCAAAACTACAGAATTACCAGATGGTATTCAAGTCC
ATGGTTTAAACTCGATAAAAGATTTGAAGGTTCCTTCCGACTCTTCTGGACCTGTCGGTGTATGCACATA
TGATGATCATCGTGTGGCCATGAGTTTCTCGCTTCTTGCAGGAATGGTAAATTCTCAAAATGAACGTGAC
GAAGTTGCTAATCCTGTAAGAATACTTGAAAGACATTGTACTGGTAAAACCTGGCCTGGCTGGTGGGATG
TGTTACATTCCGAACTAGGTGCCAAATTAGATGGTGCAGAACCTTTAGAGTGCACATCCAAAAAGAACTC
AAAGAAAAGCGTTGTCATTATTGGCATGAGAGCAGCTGGCAAAACTACTATAAGTAAATGGTGCGCATCC
GCTCTGGGTTACAAATTAGTTGACCTAGACGAGCTGTTTGAGCAACAGCATAACAATCAAAGTGTTAAAC
AATTTGTTGTGGAGAACGGTTGGGAGAAGTTCCGTGAGGAAGAAACAAGAATTTTCAAGGAAGTTATTCA
AAATTACGGCGATGATGGATATGTTTTCTCAACAGGTGGCGGTATTGTTGAAAGCGCTGAGTCTAGAAAA
GCCTTAAAAGATTTTGCCTCATCAGGTGGATACGTTTTACACTTACATAGGGATATTGAGGAGACAATTG
TCTTTTTACAAAGTGATCCTTCAAGACCTGCCTATGTGGAAGAAATTCGTGAAGTTTGGAACAGAAGGGA
GGGGTGGTATAAAGAATGCTCAAATTTCTCTTTCTTTGCTCCTCATTGCTCCGCAGAAGCTGAGTTCCAA
GCTCTAAGAAGATCGTTTAGTAAGTACATTGCAACCATTACAGGTGTCAGAGAAATAGAAATTCCAAGCG
GAAGATCTGCCTTTGTGTGTTTAACCTTTGATGACTTAACTGAACAAACTGAGAATTTGACTCCAATCTG
TTATGGTTGTGAGGCTGTAGAGGTCAGAGTAGACCATTTGGCTAATTACTCTGCTGATTTCGTGAGTAAA
CAGTTATCTATATTGCGTAAAGCCACTGACAGTATTCCTATCATTTTTACTGTGCGAACCATGAAGCAAG
GTGGCAACTTTCCTGATGAAGAGTTCAAAACCTTGAGAGAGCTATACGATATTGCCTTGAAGAATGGTGT
TGAATTCCTTGACTTAGAACTAACTTTACCTACTGATATCCAATATGAGGTTATTAACAAAAGGGGCAAC
ACCAAGATCATTGGTTCCCATCATGACTTCCAAGGATTATACTCCTGGGACGACGCTGAATGGGAAAACA
GATTCAATCAAGCGTTAACTCTTGATGTGGATGTTGTAAAATTTGTGGGTACGGCTGTTAATTTCGAAGA
TAATTTGAGACTGGAACACTTTAGGGATACACACAAGAATAAGCCTTTAATTGCAGTTAATATGACTTCT
AAAGGTAGCATTTCTCGTGTTTTGAATAATGTTTTAACACCTGTGACATCAGATTTATTGCCTAACTCCG
CTGCCCCTGGCCAATTGACAGTAGCACAAATTAACAAGATGTATACATCTATGGGAGGTATCGAGCCTAA
GGAACTGTTTGTTGTTGGAAAGCCAATTGGCCACTCTAGATCGCCAATTTTACATAACACTGGCTATGAA
ATTTTAGGTTTACCTCACAAGTTCGATAAATTTGAAACTGAATCCGCACAATTGGTGAAAGAAAAACTTT
TGGACGGAAACAAGAACTTTGGCGGTGCTGCAGTCACAATTCCTCTGAAATTAGATATAATGCAGTACAT
GGATGAATTGACTGATGCTGCTAAAGTTATTGGTGCTGTAAACACAGTTATACCATTGGGTAACAAGAAG
TTTAAGGGTGATAATACCGACTGGTTAGGTATCCGTAATGCCTTAATTAACAATGGCGTTCCCGAATATG
TTGGTCATACCGCTGGTTTGGTTATCGGTGCAGGTGGCACTTCTAGAGCCGCCCTTTACGCCTTGCACAG
TTTAGGTTGCAAAAAGATCTTCATAATCAACAGGACAACTTCGAAATTGAAGCCATTAATAGAGTCACTT
CCATCTGAATTCAACATTATTGGAATAGAGTCCACTAAATCTATAGAAGAGATTAAGGAACACGTTGGCG
TTGCTGTCAGCTGTGTACCAGCCGACAAACCATTAGATGACGAACTTTTAAGTAAGCTGGAGAGATTCCT
TGTGAAAGGTGCCCATGCTGCTTTTGTACCAACCTTATTGGAAGCCGCATACAAACCAAGCGTTACTCCC
GTTATGACAATTTCACAAGACAAATATCAATGGCACGTTGTCCCTGGATCACAAATGTTAGTACACCAAG
GTGTAGCTCAGTTTGAAAAGTGGACAGGATTCAAGGGCCCTTTCAAGGCCATTTTTGATGCCGTTACGAA
AGAGTAG
Aro1p
MVQLAKVPILGNDIIHVGYNIHDHLVETIIKHCPSSTYVICNDTNLSKVPYYQQLVLEFKASLPEGSRLLTY
VVKPGETSKSRETKAQLEDYLLVEGCTRDTVMVAIGGGVIGDMIGFVASTFMRGVRVVQVPTSLLAMVDSSI
GGKTAIDTPLGKNFIGAFWQPKFVLVDIKWLETLAKREFINGMAEVIKTACIWNADEFTRLESNASLFLNVV
NGAKNVKVTNQLTNEIDEISNTDIEAMLDHTYKLVLESIKVKAEVVSSDERESSLRNLLNFGHSIGHAYEAI
LTPQALHGECVSIGMVKEAELSRYFGILSPTQVARLSKILVAYGLPVSPDEKWFKELTLHKKTPLDILLKKM
SIDKKNEGSKKKVVILESIGKCYGDSAQFVSDEDLRFILTDETLVYPFKDIPADQQKVVIPPGSKSISNRAL
ILAALGEGQCKIKNLLHSDDTKHMLTAVHELKGATISWEDNGETVVVEGHGGSTLSACADPLYLGNAGTASR
FLTSLAALVNSTSSQKYIVLTGNARMQQRPIAPLVDSLRANGTKIEYLNNEGSLPIKVYTDSVFKGGRIELA
ATVSSQYVSSILMCAPYAEEPVTLALVGGKPISKLYVDMTIKMMEKFGINVETSTTEPYTYYIPKGHYINPS
EYVIESDASSATYPLAFAAMTGTTVTVPNIGFESLQGDARFARDVLKPMGCKITQTATSTTVSGPPVGTLKP
LKHVDMEPMTDAFLTACVVAAISHDSDPNSANTTTIEGIANQRVKECNRILAMATELAKFGVKTTELPDGIQ
VHGLNSIKDLKVPSDSSGPVGVCTYDDHRVAMSFSLLAGMVNSQNERDEVANPVRILERHCTGKTWPGWWDV
LHSELGAKLDGAEPLECTSKKNSKKSVVIIGMRAAGKTTISKWCASALGYKLVDLDELFEQQHNNQSVKQFV
VENGWEKFREEETRIFKEVIQNYGDDGYVFSTGGGIVESAESRKALKDFASSGGYVLHLHRDIEETIVFLQS
DPSRPAYVEEIREVWNRREGWYKECSNFSFFAPHCSAEAEFQALRRSFSKYIATITGVREIEIPSGRSAFVC
LTFDDLTEQTENLTPICYGCEAVEVRVDHLANYSADFVSKQLSILRKATDSIPIIFTVRTMKQGGNFPDEEF
KTLRELYDIALKNGVEFLDLELTLPTDIQYEVINKRGNTKIIGSHHDFQGLYSWDDAEWENRFNQALTLDVD
VVKFVGTAVNFEDNLRLEHFRDTHKNKPLIAVNMTSKGSISRVLNNVLTPVTSDLLPNSAAPGQLTVAQINK
MYTSMGGIEPKELFVVGKPIGHSRSPILHNTGYEILGLPHKFDKFETESAQLVKEKLLDGNKNFGGAAVTIP
LKLDIMQYMDELTDAAKVIGAVNTVIPLGNKKFKGDNTDWLGIRNALINNGVPEYVGHTAGLVIGAGGTSRA
ALYALHSLGCKKIFIINRTTSKLKPLIESLPSEFNIIGIESTKSIEEIKEHVGVAVSCVPADKPLDDELLSK
LERFLVKGAHAAFVPTLLEAAYKPSVTPVMTISQDKYQWHVVPGSQMLVHQGVAQFEKWTGFKGPFKAIFDA
VTKE
ARO2
ATGTCAACGTTTGGGAAACTGTTCCGCGTCACCACATATGGTGAATCGCATTGTAAGTCTGTCGGTTGCAT
TGTCGACGGTGTTCCTCCAGGAATGTCATTAACCGAAGCTGACATTCAGCCACAATTGACCAGAAGAAGAC
CGGGTCAATCTAAGCTATCGACCCCTAGAGACGAAAAGGATAGAGTGGAAATCCAGTCCGGTACCGAGTTC
GGCAAGACTCTAGGTACACCCATCGCCATGATGATCAAAAACGAGGACCAAAGACCTCACGACTACTCCGA
CATGGACAAGTTCCCTAGACCTTCCCATGCGGACTTCACGTACTCGGAAAAGTACGGTATCAAGGCCTCCT
CTGGTGGTGGCAGAGCTTCTGCTAGAGAAACGATTGGCCGTGTCGCTTCAGGTGCCATTGCTGAGAAGTTC
TTAGCTCAGAACTCTAATGTCGAGATCGTAGCCTTTGTGACACAAATCGGGGAAATCAAGATGAACAGAGA
CTCTTTCGATCCTGAATTTCAGCATCTGTTGAACACCATCACCAGGGAAAAAGTGGACTCAATGGGTCCTA
TCAGATGTCCAGACGCCTCCGTTGCTGGTTTGATGGTCAAGGAAATCGAAAAGTACAGAGGCAACAAGGAC
TCTATCGGTGGTGTCGTCACTTGTGTCGTGAGAAACTTGCCTACCGGTCTCGGTGAGCCATGCTTTGACAA
GTTGGAAGCCATGTTGGCTCATGCTATGTTGTCCATTCCAGCATCCAAGGGTTTCGAAATTGGCTCAGGTT
TTCAGGGTGTCTCTGTTCCAGGGTCCAAGCACAATGACCCATTTTACTTTGAAAAAGAAACAAACAGATTA
AGAACAAAGACCAACAATTCAGGTGGTGTACAAGGTGGTATCTCTAATGGTGAGAACATCTATTTCTCTGT
CCCATTCAAGTCAGTGGCCACTATCTCTCAAGAACAAAAAACCGCCACTTACGATGGTGAAGAAGGTATCT
TAGCCGCTAAGGGTAGACATGACCCTGCTGTCACTCCAAGAGCTATTCCTATTGTGGAAGCCATGACCGCT
CTGGTGTTGGCTGACGCGCTTTTGATCCAAAAGGCAAGAGATTTCTCCAGATCCGTGGTTCATTAA
Aro2p
MSTFGKLFRVTTYGESHCKSVGCIVDGVPPGMSLTEADIQPQLTRRRPGQSKLSTPRDEKDRVEIQSGTEFG
KTLGTPIAMMIKNEDQRPHDYSDMDKFPRPSHADFTYSEKYGIKASSGGGRASARETIGRVASGAIAEKFLA
QNSNVEIVAFVTQIGEIKMNRDSFDPEFQHLLNTITREKVDSMGPIRCPDASVAGLMVKEIEKYRGNKDSIG
GVVTCVVRNLPTGLGEPCFDKLEAMLAHAMLSIPASKGFEIGSGFQGVSVPGSKHNDPFYFEKETNRLRTKT
NNSGGVQGGISNGENIYFSVPFKSVATISQEQKTATYDGEEGILAAKGRHDPAVTPRAIPIVEAMTALVLAD
ALLIQKARDFSRSVVH
ABZ1
ATGCTGTCCGATACAATTGACACAAAGCAACAACAGCAACAGCTTCATGTCCTGTTCATAGACTCTTATGA
TTCATTCACCTACAATGTAGTGAGACTAATTGAACAACAAACTGATATCTCACCGGGAGTCAACGCCGTGC
ACGTGACGACGGTACATAGTGATACGTTCCAATCTATGGATCAGCTATTGCCACTTTTGCCGCTTTTTGAT
GCTATCGTTGTTGGCCCAGGACCTGGGAATCCCAACAATGGTGCACAAGATATGGGTATAATATCTGAGCT
TTTCGAGAATGCCAATGGAAAGTTAGATGAAGTTCCAATATTGGGTATATGTCTTGGGTTCCAAGCAATGT
GCTTGGCTCAAGGTGCTGATGTCAGTGAGCTAAATACTATCAAGCATGGGCAAGTGTATGAAATGCATTTA
AACGATGCAGCCAGAGCTTGTGGCCTTTTTTCTGGTTATCCCGATACGTTCAAATCTACGAGGTACCATTC
ATTGCATGTCAATGCCGAAGGCATTGACACCCTTTTGCCCTTATGCACAACCGAAGATGAGAACGGTATTC
TTTTGATGAGTGCTCAAACGAAAAATAAGCCATGGTTTGGCGTACAGTACCACCCGGAGTCATGTTGTTCA
GAATTGGGGGGGCTGTTAGTCAGTAACTTTCTCAAGTTGAGTTTCATAAATAACGTGAAGACAGGAAGGTG
GGAAAAGAAGAAACTTAATGGAGAGTTTTCCGATATCCTATCTCGATTGGATAGGACTATTGATAGAGACC
CCATATACAAGGTAAAAGAGAAATATCCGAAGGGCGAGGACACAACTTACGTTAAGCAGTTCGAGGTCTCT
GAAGACCCGAAATTGACATTTGAAATTTGCAACATCATACGAGAAGAAAAATTTGTCATGTCATCTTCTGT
GATTAGTGAAAATACGGGTGAATGGTCTATCATTGCTTTACCAAACTCCGCATCCCAGGTATTCACTCATT
ATGGAGCTATGAAAAAGACTACAGTTCATTATTGGCAAGATAGTGAAATTAGTTACACCTTGTTGAAAAAG
TGTCTAGATGGTCAAGATTCGGATTTGCCTGGCTCCCTTGAGGTAATACATGAAGATAAATCCCAATTTTG
GATCACTTTGGGTAAATTTATGGAGAATAAAATAATCGATAACCACAGAGAAATACCTTTTATTGGAGGTC
TTGTTGGCATTTTAGGTTATGAAATAGGTCAGTACATTGCATGCGGCCGTTGCAATGATGATGAGAATTCC
CTTGTTCCCGACGCCAAACTAGTTTTTATCAACAATAGTATAGTCATTAATCACAAGCAAGGGAAGCTTTA
TTGTATTTCTCTGGATAATACATTTCCAGTGGCATTAGAACAATCATTAAGGGACAGTTTTGTTAGAAAGA
AGAATATTAAGCAATCCCTGTCCTGGCCCAAGTATCTTCCAGAGGAGATAGACTTCATTATAACTATGCCC
GATAAACTTGACTACGCTAAGGCGTTTAAGAAATGTCAGGATTATATGCATAAGGGTGATTCTTATGAAAT
GTGTCTCACAACGCAAACCAAAGTTGTACCATCTGCGGTGATAGAACCCTGGAGGATTTTCCAGACCTTGG
TACAAAGAAACCCGGCTCCATTTTCAAGTTTTTTTGAGTTTAAAGACATTATTCCCCGCCAAGATGAAACG
CCTCCAGTTTTGTGCTTCTTAAGTACTTCTCCAGAAAGGTTTTTGAAGTGGGATGCAGACACATGCGAGCT
ACGTCCCATCAAGGGAACTGTGAAAAAAGGACCGCAAATGAACTTGGCAAAAGCCACACGAATCCTGAAGA
CACCAAAAGAATTTGGTGAGAACTTAATGATTTTGGACTTAATCAGAAATGACCTTTACGAGTTGGTTCCT
GACGTTCGGGTGGAGGAGTTCATGTCCGTGCAAGAATATGCCACCGTTTACCAACTCGTTAGCGTCGTAAA
GGCACATGGATTGACCTCTGCCAGTAAGAAGACGAGATATTCAGGCATTGATGTCCTTAAACACTCGCTTC
CTCCGGGATCTATGACGGGAGCCCCCAAGAAGATTACTGTGCAATTATTGCAGGACAAGATAGAAAGCAAG
CTAAACAAACATGTCAATGGTGGAGCACGTGGTGTTTACAGCGGTGTCACGGGATATTGGTCTGTGAATTC
CAACGGAGATTGGTCTGTTAACATTAGATGTATGTATTCCTACAACGGCGGAACCAGCTGGCAACTCATGT
GGTGCAGGGGGGGCCATAACAGTCTTAAGCACACTAGATGGCGAACTAGAGGAAACAACAAGTTGGAGAGC
AACTTACAAATTTTCATGTAG
Abz1p
MLSDTIDTKQQQQQLHVLFIDSYDSFTYNVVRLIEQQTDISPGVNAVHVTTVHSDTFQSMDQLLPLLPLFD
AIVVGPGPGNPNNGAQDMGIISELFENANGKLDEVPILGICLGFQAMCLAQGADVSELNTIKHGQVYEMHL
NDAARACGLFSGYPDTFKSTRYHSLHVNAEGIDTLLPLCTTEDENGILLMSAQTKNKPWFGVQYHPESCCS
ELGGLLVSNFLKLSFINNVKTGRWEKKKLNGEFSDILSRLDRTIDRDPIYKVKEKYPKGEDTTYVKQFEVS
EDPKLTFEICNIIREEKFVMSSSVISENTGEWSIIALPNSASQVFTHYGAMKKTTVHYVVQDSEISYTLLK
KCLDGQDSDLPGSLEVIHEDKSQFWITLGKFMENKIIDNHREIPFIGGLVGILGYEIGQYIACGRCNDDEN
SLVPDAKLVFINNSIVINHKQGKLYCISLDNTFPVALEQSLRDSFVRKKNIKQSLSWPKYLPEEIDFIITM
PDKLDYAKAFKKCQDYMHKGDSYEMCLTTQTKVVPSAVIEPWRIFQTLVQRNPAPFSSFFEFKDIIPRQDE
TPPVLCFLSTSPERFLKWDADTCELRPIKGTVKKGPQMNLAKATRILKTPKEFGENLMILDLIRNDLYELV
PDVRVEEFMSVQEYATVYQLVSVVKAHGLTSASKKTRYSGIDVLKHSLPPGSMTGAPKKITVQLLQDKIES
KLNKHVNGGARGVYSGVTGYVVSVNSNGDWSVNIRCMYSYNGGTSWQLGAGGAITVLSTLDGELEEMYNKL
ESNLQIFM
ABZ2
ATGTCACTAATGGACAATTGGAAGACTGATATGGAAAGTTACGATGAAGGAGGCCTAGTTGCTAATCCGA
ACTTCGAGGTTCTGGCCACTTTCAGGTACGACCCTGGTTTTGCACGCCAGTCAGCGTCAAAGAAAGAGAT
CTTTGAAACTCCAGACCCTCGATTAGGTTTGAGAGACGAAGATATTAGGCAGCAGATAATTAATGAGGAT
TACTCAAGTTATTTACGAGTAAGGGAGGTTAATTCCGGCGGTGACCTTCTCGAAAATATTCAGCATCCTGA
TGCTTGGAAGCATGATTGCAAGACCATTGTGTGCCAGCGTGTAGAAGATATGCTACAAGTCATTTATGAA
CGATTTTTTTTATTAGATGAACAATACCAAAGAATAAGAATAGCATTATCATACTTTAAAATTGACTTCA
GCACGTCTCTGAATGATTTATTGAAGTTATTGGTTGAAAACTTGATTAATTGTAAAGAAGGAAATTCAGA
GTATCACGAAAAAATTCAAAAAATGATCAACGAAAGGCAATGCTATAAAATGCGGGTACTTGTCTCTAAG
ACAGGAGATATACGAATTGAGGCAATTCCAATGCCTATGGAGCCTATCCTAAAATTAACAACCGATTATG
ACAGTGTTTCCACATACTTCATCAAAACGATGCTCAATGGATTTTTAATTGATAGCACAATAAATTGGGA
TGTTGTTGTTTCATCTGAACCATTGAACGCATCAGCTTTCACCAGTTTTAAAACCACTTCAAGAGATCATT
ACGCTAGGGCGAGAGTTCGCATGCAAACTGCTATAAATAACTTAAGAGGTTCAGAACCTACTTCTTCTGTC
TCGCAATGCGAAATTTTATTTTCCAACAAATCTGGCCTGCTGATGGAAGGTTCAATAACAAACGTGGCTG
TAATTCAAAAAGATCCTAACGGTTCTAAAAAGTATGTGACACCAAGATTAGCAACTGGATGTTTGTGCGG
AACAATGCGTCATTATTTATTGCGGCTCGGCCTTATTGAAGAGGGAGATATAGATATAGGAAGCCTTACC
GTTGGCAACGAAGTTTTGCTTTTCAATGGCGTCATGGGATGCATAAAGGGAACAGTGAAGACAAAATATT
GA
Abz2p
MSLMDNWKTDMESYDEGGLVANPNFEVLATFRYDPGFARQSASKKEIFETPDPRLGLRDEDIRQQIINEDY
SSYLRVREVNSGGDLLENIQHPDAWKHDCKTIVCQRVEDMLQVIYERFFLLDEQYQRIRIALSYFKIDFSTSL
NDLLKLLVENLINCKEGNSEYHEKIQKMINERQCYKMRVLVSKTGDIRIEAIPMPMEPILKLTTDYDSVSTYF
IKTMLNGFLIDSTINWDVVVSSEPLNASAFTSFKTTSRDHYARARVRMQTAINNLRGSEPTSSVSQCEILFSN
KSGLLMEGSITNVAVIQKDPNGSKKYVTPRLATGCLCGTMRHYLLRLGLIEEGDIDIGSLTVGNEVLLFNGV
MGCIKGTVKTKY
FOL1
ATGTCAAAGCTATTTTCTACTGTCAATTCTGCAAGACATAGTGTACCACTAGGCGGCATGAGAGATTATG
TGCACATTAAGAAACTAGAGATGAATACAGTTCTTGGGCCTGATTCCTGGAATCAATTAATGCCTCAGAA
ATGTCTACTAAGCTTAGATATGGGTACAGATTTTAGTAAATCTGCGGCTACGGATGATTTGAAATATTCT
CTAAATTATGCAGTTATTTCTCGTGATTTGACGAATTTCGTCAGCAAAAAAAAGAATTGGGGTTCTGTTT
CTAATTTGGCTAAATCTGTGTCTCAATTTGTTATGGACAAATATTCTGGTGTCGAGTGTCTGAATTTAGA
AGTGCAGGCGGATACAACGCATATTAGAAGTGACCACATATCTTGTATTATTCAACAAGAAAGAGGGAAT
CCAGAATCACAGGAATTTGACGTTGTTAGGATATCTGAGTTAAAAATGTTGACTTTGATTGGTGTTTTCA
CCTTTGAGAGACTTAAGAAACAGTATGTAACTTTGGATATAAAGTTGCCTTGGCCAAAGAAAGCCGAATT
GCCACCGCCAGTGCAAAGCATAATTGATAACGTTGTCAAGTTTGTGGAGGAATCAAATTTCAAGACTGTG
GAAGCTCTTGTAGAATCTGTGTCAGCTGTTATTGCCCATAACGAGTATTTTCAAAAGTTTCCAGATTCGCC
TTTGGTGGTGAAGGTTTTGAAATTAAACGCAATCACAGCCACAGAAGGTGTTGGTGTAAGCTGTATTAGA
GAGCCCAGGGAGATTGCGATGGTAAATATTCCATATCTTTCCTCCATACATGAATCGTCTGATATTAAGTT
CCAATTGTCTTCATCACAAAACACTCCTATTGAGGGTAAAAATACATGGAAAAGAGCGTTTTTAGCGTTT
GGTTCAAACATTGGGGACCGTTTCAAACACATTCAAATGGCGTTGCAATTATTATCAAGGGAAAAAACGG
TTAAATTACGGAATATTTCGTCTATTTTTGAAAGTGAACCAATGTATTTCAAAGATCAAACCCCTTTCAT
GAATGGGTGTGTTGAGGTGGAGACATTACTGACCCCAAGCGAATTATTAAAATTGTGTAAAAAAATTGAA
TATGAAGAGTTGCAAAGAGTCAAGCATTTTGATAATGGTCCGAGAACAATAGATCTGGATATTGTTATGT
TTTTGAATAGCGCCGGAGAAGATATTATAGTAAATGAACCGGATTTGAATATACCGCATCCTAGAATGCT
GGAGAGGACTTTCGTTCTTGAGCCGTTATGTGAATTAATATCCCCCGTTCACCTTCATCCTGTGACAGCGG
AACCCATTGTAGACCATTTAAAACAGTTATACGACAAACAGCATGATGAAGATACCTTATGGAAATTAGT
TCCATTGCCTTATCGTAGTGGTGTGGAGCCTAGATTTTTGAAATTCAAGACCGCTACAAAACTTGACGAAT
TTACTGGAGAAACAAACAGAATTACTGTTTCACCTACATATATCATGGCTATCTTCAACGCTACACCAGAT
TCATTTTCCGATGGAGGTGAGCATTTTGCGGACATTGAAAGTCAATTGAATGATATCATTAAATTGTGTA
AAGACGCATTATATTTGCATGAGAGCGTCATCATCGACGTTGGAGGGTGTTCTACCAGGCCTAACTCTATT
CAGGCGTCTGAGGAAGAAGAAATACGCAGGTCTATCCCATTAATTAAGGCCATTAGAGAAAGCACTGAGT
TACCGCAAGATAAAGTCATACTATCCATTGATACTTATCGTTCCAATGTCGCTAAAGAAGCGATTAAAGT
TGGAGTGGATATTATTAATGATATTTCGGGAGGTTTATTTGACAGCAACATGTTTGCCGTAATTGCAGAG
AACCCAGAAATTTGTTATATTTTATCACACACACGTGGTGATATTTCAACGATGAATAGGCTGGCGCATT
ACGAAAATTTTGCATTGGGTGATTCTATTCAGCAAGAATTTGTTCATAATACCGACATTCAGCAGCTAGA
CGACTTGAAAGACAAAACAGTGTTAATCAGGAATGTTGGTCAAGAAATTGGCGAAAGGTATATCAAAGCG
ATTGATAATGGAGTAAAGCGCTGGCAAATTCTAATCGACCCTGGACTTGGTTTTGCTAAGACCTGGAAGC
AAAACTTACAAATTATTAGACATATCCCCATTTTAAAGAACTACTCATTCACCATGAACTCAAACAATTC
GCAAGTGTATGTTAACCTCAGAAATATGCCCGTTTTATTGGGTCCATCGCGCAAAAAATTCATTGGACAT
ATCACAAAAGATGTGGATGCGAAGCAAAGAGACTTTGCTACTGGAGCGGTGGTAGCGTCGTGTATTGGTT
TCGGCAGCGACATGGTTAGGGTCCATGACGTTAAAAATTGTTCGAAGAGCATTAAATTAGCAGATGCTAT
TTATAAAGGTTTGGAATAA
Fol1p
MSKLFSTVNSARHSVPLGGMRDYVHIKKLEMNTVLGPDSWNQLMPQKCLLSLDMGTDFSKSAATDDLKYS
LNYAVISRDLTNFVSKKKNWGSVSNLAKSVSQFVMDKYSGVECLNLEVQADTTHIRSDHISCIIQQERGNPE
SQEFDVVRISELKMLTLIGVFTFERLKKQYVTLDIKLPWPKKAELPPPVQSIIDNVVKFVEESNFKTVEALVE
SVSAVIAHNEYFQKFPDSPLVVKVLKLNAITATEGVGVSCIREPREIAMVNIPYLSSIHESSDIKFQLSSSQNTP
IEGKNTWKRAFLAFGSNIGDRFKHIQMALQLLSREKTVKLRNISSIFESEPMYFKDQTPFMNGCVEVETLLT
PSELLKLCKKIEYEELQRVKHFDNGPRTIDLDIVMFLNSAGEDIIVNEPDLNIPHPRMLERTFVLEPLCELISP
VHLHPVTAEPIVDHLKQLYDKQHDEDTLWKLVPLPYRSGVEPRFLKFKTATKLDEFTGETNRITVSPTYIM
AIFNATPDSFSDGGEHFADIESQLNDIIKLCKDALYLHESVIIDVGGCSTRPNSIQASEEEEIRRSIPLIKAIRES
TELPQDKVILSIDTYRSNVAKEAIKVGVDIINDISGGLFDSNMFAVIAENPEICYILSHTRGDISTMNRLAHYE
NFALGDSIQQEFVHNTDIQQLDDLKDKTVLIRNVGQEIGERYIKAIDNGVKRWQILIDPGLGFAKTWKQNLQ
IIRHIPILKNYSFTMNSNNSQVYVNLRNMPVLLGPSRKKFIGHITKDVDAKQRDFATGAVVASCIGFGSDMV
RVHDVKNCSKSIKLADAIYKGLE
TRP1
ATGTCTGTTATTAATTTCACAGGTAGTTCTGGTCCATTGGTGAAAGTTTGCGGCTTGCAGAGCACAGAGG
CCGCAGAATGTGCTCTAGATTCCGATGCTGACTTGCTGGGTATTATATGTGTGCCCAATAGAAAGAGAAC
AATTGACCCGGTTATTGCAAGGAAAATTTCAAGTCTTGTAAAAGCATATAAAAATAGTTCAGGCACTCCG
AAATACTTGGTTGGCGTGTTTCGTAATCAACCTAAGGAGGATGTTTTGGCTCTGGTCAATGATTACGGCA
TTGATATCGTCCAACTGCATGGAGATGAGTCGTGGCAAGAATACCAAGAGTTCCTCGGTTTGCCAGTTATT
AAAAGACTCGTATTTCCAAAAGACTGCAACATACTACTCAGTGCAGCTTCACAGAAACCTCATTCGTTTAT
TCCCTTGTTTGATTCAGAAGCAGGTGGGACAGGTGAACTTTTGGATTGGAACTCGATTTCTGACTGGGTT
GGAAGGCAAGAGAGCCCCGAAAGCTTACATTTTATGTTAGCTGGTGGACTGACGCCAGAAAATGTTGGTG
ATGCGCTTAGATTAAATGGCGTTATTGGTGTTGATGTAAGCGGAGGTGTGGAGACAAATGGTGTAAAAGA
CTCTAACAAAATAGCAAATTTCGTCAAAAATGCTAAGAAATAG
Trp1p
MSVINFTGSSGPLVKVCGLQSTEAAECALDSDADLLGIICVPNRKRTIDPVIARKISSLVKAYKNSSGTPKY
LVGVFRNQPKEDVLALVNDYGIDIVQLHGDESWQEYQEFLGLPVIKRLVFPKDCNILLSAASQKPHSFIPLF
DSEAGGTGELLDWNSISDWVGRQESPESLHFMLAGGLTPENVGDALRLNGVIGVDVSGGVETNGVKDSNKIA
NFVKNAKK
TRP2
ATGACCGCTTCCATCAAAATTCAACCGGATATTGACTCTCTAAAGCAATTACAGCAGCAAAATGACGATA
GTTCCATAAATATGTATCCCGTGTATGCGTATTTGCCATCATTGGATCTGACTCCTCACGTGGCATATCTA
AAATTGGCACAATTGAACAACCCTGATAGAAAGGAATCATTTCTGTTGGAAAGTGCTAAGACAAATAATG
AATTAGATCGTTATTCATTCATAGGTATCTCGCCACGCAAGACCATCAAAACCGGTCCTACTGAAGGCATT
GAAACAGATCCTTTGGAAATTTTGGAAAAGGAGATGTCTACCTTTAAAGTAGCCGAAAATGTTCCTGGTT
TACCGAAATTAAGTGGTGGTGCTATTGGTTATATTTCTTATGACTGTGTTCGTTATTTCGAGCCAAAAAC
AAGAAGGCCTTTGAAAGATGTCCTAAGACTTCCAGAGGCATATTTAATGCTTTGTGATACCATTATTGCC
TTTGATAATGTTTTTCAGAGATTTCAAATCATTCATAACATTAATACCAATGAAACTTCGTTGGAGGAAG
GTTACCAAGCTGCAGCACAAATAATCACTGATATCGTATCAAAGCTAACCGACGATTCCTCGCCAATACCA
TATCCAGAACAACCTCCTATTAAATTGAATCAAACTTTTGAATCGAATGTGGGCAAGGAAGGTTACGAAA
ATCACGTCTCCACTTTGAAGAAGCATATTAAGAAAGGTGATATTATTCAAGGTGTGCCATCGCAAAGAGT
GGCAAGGCCAACTTCGTTACATCCTTTCAATATTTACAGACATTTACGTACAGTGAACCCATCTCCTTACC
TGTTTTATATTGATTGTTTGGATTTCCAAATCATTGGTGCATCTCCAGAATTGTTGTGCAAATCGGATTCC
AAAAATAGAGTCATTACCCATCCAATTGCTGGTACTGTCAAACGTGGGGCTACTACTGAAGAGGATGATG
CTTTAGCGGACCAATTACGTGGCTCGTTAAAAGACCGTGCAGAACATGTTATGCTGGTAGATTTAGCAAG
AAACGATATTAACAGAATTTGTGACCCATTAACAACAAGTGTCGATAAACTGTTAACTATTCAAAAATTT
TCTCATGTCCAACATCTGGTTTCTCAAGTCAGCGGTGTTCTCCGCCCAGAAAAGACAAGATTTGATGCATT
CAGATCGATTTTCCCTGCAGGTACTGTCAGTGGTGCTCCAAAGGTTAGAGCCATGGAATTGATTGCCGAAC
TAGAAGGAGAAAGGCGTGGGGTTTATGCAGGCGCCGTAGGTCATTGGTCATACGACGGTAAAACAATGGA
CAATTGTATCGCTTTAAGGACTATGGTCTATAAAGATGGCATTGCTTACTTGCAAGCTGGCGGTGGTATT
GTTTACGATTCAGATGAGTACGATGAATATGTCGAAACCATGAATAAAATGATGGCCAATCACAGTACTA
TTGTGCAAGCAGAAGAATTGTGGGCCGATATCGTAGGATCAGCTTAA
Trp2p
MTASIKIQPDIDSLKQLQQQNDDSSINMYPVYAYLPSLDLTPHVAYLKLAQLNNPDRKESFLLESAKTNNEL
DRYSFIGISPRKTIKTGPTEGIETDPLEILEKEMSTFKVAENVPGLPKLSGGAIGYISYDCVRYFEPKTRRP
LKDVLRLPEAYLMLCDTIIAFDNVFQRFQIIHNINTNETSLEEGYQAAAQIITDIVSKLTDDSSPIPYPEQP
PIKLNQTFESNVGKEGYENHVSTLKKHIKKGDIIQGVPSQRVARPTSLHPFNIYRHLRTVNPSPYLFYIDCL
DFQIIGASPELLCKSDSKNRVITHPIAGTVKRGATTEEDDALADQLRGSLKDRAEHVMLVDLARNDINRICD
PLTTSVDKLLTIQKFSHVQHLVSQVSGVLRPEKTRFDAFRSIFPAGTVSGAPKVRAMELIAELEGERRGVYA
GAVGHWSYDGKTMDNCIALRTMVYKDGIAYLQAGGGIVYDSDEYDEYVETMNKMMANHSTIVQAEELWADIV
GSA
PHA2
ATGGCCAGCAAGACTTTGAGGGTTCTTTTTCTGGGTCCCAAAGGTACGTATTCCCATCAAGCTGCATTACA
ACAATTTCAATCAACATCTGATGTTGAGTACCTCCCAGCAGCCTCTATCCCCCAATGTTTTAACCAATTGG
AGAACGACACTAGTATAGATTATTCAGTGGTACCGTTGGAAAATTCCACCAATGGACAAGTAGTTTTTTC
CTATGATCTCTTGCGTGATAGGATGATCAAAAAAGCCCTATCCTTACCTGCTCCAGCAGATACTAATAGAA
TTACACCAGATATAGAAGTTATAGCGGAGCAATATGTACCCATTACCCATTGTCTAATCAGCCCAATCCAA
CTACCAAATGGTATTGCATCCCTTGGAAATTTTGAAGAAGTCATAATACACTCACATCCGCAAGTATGGG
GCCAGGTTGAATGTTACTTAAGGTCCATGGCAGAAAAATTTCCGCAGGTCACCTTTATAAGATTGGATTG
TTCTTCCACATCTGAATCAGTGAACCAATGCATTCGGTCATCAACGGCCGATTGCGACAACATTCTGCATT
TAGCCATTGCTAGTGAAACAGCTGCCCAATTGCATAAGGCGTACATCATTGAACATTCGATAAATGATAA
GCTAGGAAATACAACAAGATTTTTAGTATTGAAGAGAAGGGAGAACGCAGGCGACAATGAAGTAGAAGAC
ACTGGATTACTACGGGTTAACCTACTCACCTTTACTACTCGTCAAGATGACCCTGGTTCTTTGGTAGATGT
TTTGAACATACTAAAAATCCATTCACTCAACATGTGTTCTATAAACTCTAGACCATTCCATTTGGACGAAC
ATGATAGAAACTGGCGATATTTATTTTTCATTGAATATTACACCGAGAAGAATACCCCAAAGAATAAAGA
AAAATTCTATGAAGATATCAGCGACAAAAGTAAACAGTGGTGCCTGTGGGGTACATTCCCCAGAAATGAG
AGATATTATCACAAATAA
Pha2p
MASKTLRVLFLGPKGTYSHQAALQQFQSTSDVEYLPAASIPQCFNQLENDTSIDYSVVPLENSTNGQVVFSY
DLLRDRMIKKALSLPAPADTNRITPDIEVIAEQYVPITHCLISPIQLPNGIASLGNFEEVIIHSHPQVWGQVEC
YLRSMAEKFPQVTFIRLDCSSTSESVNQCIRSSTADCDNILHLAIASETAAQLHKAYIIEHSINDKLGNTTRFL
VLKRRENAGDNEVEDTGLLRVNLLTFTTRQDDPGSLVDVLNILKIHSLNMCSINSRPFHLDEHDRNWRYLF
FIEYYTEKNTPKNKEKFYEDISDKSKQWCLWGTFPRNERYYHK
ARO7
ATGGATTTCACAAAACCAGAAACTGTTTTAAATCTACAAAATATTAGAGATGAATTAGTTAGAATGGAGG
ATTCGATCATCTTCAAATTTATTGAGAGGTCGCATTTCGCCACATGTCCTTCAGTTTATGAGGCAAACCAT
CCAGGTTTAGAAATTCCGAATTTTAAAGGATCTTTCTTGGATTGGGCTCTTTCAAATCTTGAAATTGCGC
ATTCTCGCATCAGAAGATTCGAATCACCTGATGAAACTCCCTTCTTTCCTGACAAGATTCAGAAATCATTC
TTACCGAGCATTAACTACCCACAAATTTTGGCGCCTTATGCCCCAGAAGTTAATTACAATGATAAAATAA
AAAAAGTTTATATTGAAAAGATTATACCATTAATTTCGAAAAGAGATGGTGATGATAAGAATAACTTCGG
TTCTGTTGCCACTAGAGATATAGAATGTTTGCAAAGCTTGAGTAGGAGAATCCACTTTGGCAAGTTTGTT
GCTGAAGCCAAGTTCCAATCGGATATCCCGCTATACACAAAGCTGATCAAAAGTAAAGATGTCGAGGGGA
TAATGAAGAATATCACCAATTCTGCCGTTGAAGAAAAGATTCTAGAAAGATTAACTAAGAAGGCTGAAGT
CTATGGTGTGGACCCTACCAACGAGTCAGGTGAAAGAAGGATTACTCCAGAATATTTGGTAAAAATTTAT
AAGGAAATTGTTATACCTATCACTAAGGAAGTTGAGGTGGAATACTTGCTAAGAAGGTTGGAAGAGTAA
Aro7p
MDFTKPETVLNLQNIRDELVRMEDSIIFKFIERSHFATCPSVYEANHPGLEIPNFKGSFLDWALSNLEIAHSR
IRRFESPDETPFFPDKIQKSFLPSINYPQILAPYAPEVNYNDKIKKVYIEKIIPLISKRDGDDKNNFGSVATR
DIECLQSLSRRIHFGKFVAEAKFQSDIPLYTKLIKSKDVEGIMKNITNSAVEEKILERLTKKAEVYGVDPTNE
SGERRITPEYLVKIYKEIVIPITKEVEVEYLLRRLEE
4-Aminobenzoate 1-monooxygenase gene
ATGTCTCAACAGGAGCGCACCCGCGTGGCCATTGTTGGCGCAGGCATTGTTGGCCTCACTCTGGCGATTGC
TCTTAACGCTTTCGATAAGGAGCGTAAACTGGCCATCGATATTTATGAGAATGCTTCTGAACTCGCTGAA
ATCGGCGCCGGTATCAACGTTTGGCCCAGAACATTGGCAATCTTCAAACAAATCGGCGTCGAGGATGCTCT
CATTCCTCTGCTCGATCACATTCCCGACCTCGAACCACGAATTATCTTTGGCATACGGAAAGGAGACGAGA
AGAACGGATACCAAGTCTATGATACCATGAACAACGGTGGTGCCCTCCGTGTACACAGAGCTCATCTTCAG
AACACTCTTATCCAACATCTACCTCTGCCAGGCTCGAAAGTCACAGAAATCAATAGCATCTGTGGTTTCCA
TTTAGGGCACAATCTCATTGACTATAGTCATCACTCTTCATCAGGCCAAGGTCCTCTCACCCTCCATTTCT
CTGACGGAAAGCCATCCAGGACATGTGACATTCTTGTTGGCGCTGACGGGATTAAATCAACACTCCGCCAC
CTGTTTTTGCCCAGGTTACCGAATCCGGAGAAGTATCTGAACTGTTACGAGCCCAAGTGGAAAGGACTTTT
GGCGTATCGCGGTCTTGTTCCCAAGGAAAAGCTAGAAGCAGTCTCTCCTGGGCATAGAGCTCTTACTCATC
CTGGGCTCATGTATAGCGGAAAAAGCGCCTACGCCGTCGTTTATCCTGTCTCCAACGGAAAGTTTATCAAC
GTTGTTGCTATCGTTCACGACAATCCCACAAACTCAACTGTATGGCCGGGACCATGGAGAATGGATGTAAC
CCAAAGCGAATTTTTTGAAGTATACAAGGGCTGGGACGAGGAAGTCCTGGATCTCATCCGCTGTGTCGAT
AAACCAACTAAATGGGCACTCCATGCTCTGGATCATTTGGATGTCTACGCAAAGGGGAGAGTCTTCTTGAT
GGGTGATGCCGCACATGCAATGCTTCCGCATCTTGGGGCAGGAGCACACGTTGGTATGGAGGACGCATACA
TCCTTGCCTCTCTGATCACACATTCTTCGACTCCTATCTGGCCCTCAACGCAACATGTCAGCGAAATTGCC
AATATTTATAATACGATGCGTATTCCGCGGGCTGTCTCAATGTCCAATTCGACCGACGAAGCAGGCTATCT
CTGTAATTTAGAAAATCCTGGACTCGAAGAATTCAAGGTCGGAGATCACATTCCCAAAGAACTCTTAATT
CAGACAGCTCGTACCATGGAGAAGAAGTGGGCGTGGACAACTACGTACGCGGATGAGGATAGGATTAAGG
CGATTTCGCTGCTTGAAGGGCCTAGAGCGGTGCTATAA
4-Aminobenzoate 1-monooxygenase protein
MSQQERTRVAIVGAGIVGLTLAIALNAFDKERKLAIDIYENASELAEIGAGINVWPRTLAIFKQIGVEDALIPL
LDHIPDLEPRIIFGIRKGDEKNGYQVYDTMNNGGALRVHRAHLQNTLIQHLPLPGSKVTEINSICGFHLGHN
LIDYSHHSSSGQGPLTLHFSDGKPSRTCDILVGADGIKSTLRHLFLPRLPNPEKYLNCYEPKWKGLLAYRGLV
PKEKLEAVSPGHRALTHPGLMYSGKSAYAVVYPVSNGKFINVVAIVHDNPTNSTVWPGPWRMDVTQSEFF
EVYKGWDEEVLDLIRCVDKPTKWALHALDHLDVYAKGRVFLMGDAAHAMLPHLGAGAHVGMEDAYILAS
LITHSSTPIWPSTQHVSEIANIYNTMRIPRAVSMSNSTDEAGYLCNLENPGLEEFKVGDHIPKELLIQTARTM
EKKWAWTTTYADEDRIKAISLLEGPRAVL

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A recombinant microbial host cell capable of converting a fermentable carbon substrate to p-aminobenzoic acid biologically.

2. The recombinant microbial host cell of claim 1, wherein the microbial host cell is a bacterium, a cyanobacterium, an archaeon, or a fungus.

3. The recombinant microbial host cell of claim 1, wherein the microbial host cell is Escherichia coli.

4. The recombinant microbial host cell of claim 2, wherein the microbial host cell is a Gram positive bacterium or a filamentous fungus.

5. (canceled)

6. (canceled)

7. The recombinant microbial host cell of claim 1, wherein the microbial host cell is Saccharomyces cerevisiae, Kluyveromyces lactis, Aspergillus niger or Synechocystis sp. Strain PCC 6803.

8-11. (canceled)

12. A method for fermentative production of p-aminobenzoic acid comprising converting a fermentable carbon substrate to p-aminobenzoic acid by biological fermentation using a recombinant microbial host cell.

13. The method of claim 12, wherein the recombinant microbial host cell is E. coli, wherein the recombinant E. coli host cell is characterized by an inactivated 7,8-dihyropteroate synthase by mutation or enzymatic inhibition thereby preventing conversion of p-aminobenzoic acid to 7,8-dihyropteroate.

14. (canceled)

15. The method of claim 13, wherein the recombinant E. coli host cell is a 7,8-dihyropteroate synthase mutant requiring supplementation of methionine, glycine, thymidine, and pantothenate to maintain cell viability, wherein the 7,8-dihyropteroate synthase mutant is rescued with folic acid transporters from Arabidopsis thaliana or Synechocystis sp. PCC6803 in the presence of (6R,6S)-5-formyl-tetrahydrofolic acid or folic acid.

16-18. (canceled)

19. The method of claim 14, wherein the recombinant E. coli host cell is characterized by a mutated anthranilate synthase with altered enzymatic activity that catalyses production of p-aminobenzoic acid is used in place of the aminodeoxychorismate synthase and 4-amino-4-deoxychorismate lyase activities.

20. The method of claim 12, wherein the recombinant microbial host cell is S. cerevisiae, wherein the recombinant S. cerevisiae host cell is characterized by an inactivated the 7,8-dihyropteroate synthase activity by mutation or enzymatic inhibitors to prevent further conversion of p-aminobenzoic acid to 7,8-dihyropteroate.

21. (canceled)

22. The method of claim 20, wherein the recombinant S. cerevisiae host cell is a 7,8-dihyropteroate synthase mutant requiring supplementation of (6R,6S)-5-formyl-tetrahydrofolic acid or folic acid, wherein the 7,8-dihyropteroate synthase mutant is characterized by increased activities of aminodeoxychorismate synthase and 4-amino-4-deoxychorismate lyase activities by overexpression of corresponding genes that enhance conversion of chorismic acid to p-aminobenzoic acid.

23-61. (canceled)

62. A method for making p-phenylenediamines comprising reacting biologically-derived p-aminophenol (PAP) of claim 35 and ammonia in the presence of a precious metal catalyst on a support.

63-72. (canceled)

73. A method for making aniline comprising decarboxylating p-aminobenzoic acid, wherein the p-aminobenzoic acid is prepared from fermentation using a recombinant microbial host cell capable of converting a fermentable carbon substrate to p-aminobenzoic acid biologically.

74. The method of claim 73 wherein the decarboxylation is carried out thermally by heating in a solution or neat in a melt.

75. The method of claim 73, wherein the decarboxylation is carried out thermally in the presence of an acid catalyst.

76. The method of claim 74, wherein the solution is made by dissolving p-aminobenzoic acid in water or in a thermally stable organic solvent.

77-85. (canceled)

86. The method of claim 74, further comprising treating aniline with formaldehyde in water in the presence of a catalyst to produce methylenedianiline and/or poly-methylenedianiline.

87. The method of claim 86, wherein the formaldehyde is produced from an organic carbon source, and wherein the formaldehyde is produced by catalytic dehydration of fermentation-derived methanol.

88-94. (canceled)

95. The method of claim 86, further comprising converting methylenedianiline and poly-methylenedianiline to the corresponding isocyanates, including methylene diphenyl diisocyanate and poly-methylene diphenyl diisocyanate, wherein the methylene diphenyl diisocyanate and poly-methylene diphenyl diisocyanate are prepared from biologically-derived methylenedianiline and biologically-derived poly-methylenedianiline, respectively.

96. (canceled)

97. The method of claim 95, comprising reacting methylenedianiline or poly-methylenedianiline with phosgene in an inert solvent to produce methylene diphenyl diisocyanate or poly-methylene diphenyl diisocyanate.

98-100. (canceled)

101. The method of claim 95, further comprising distilling methylene diphenyl diisocyanate and fractionally distilling methylene diphenyl diisocyanate.

102. (canceled)

103. (canceled)

104. The method of claim 95, further comprising reacting methylene diphenyl diisocyanate or poly-methylene diphenyl diisocyanate with polyols or polyesterdiols to produce polyurethane polymers and prepolymers, wherein the methylene diphenyl diisocyanate and poly-methylene diphenyl diisocyanate are partially or totally biologically-derived and the polyols and polyesterdiols are prepared from biologically sourced ethylene glycol, propanediol, butanediol, hexanediol, adipic acid, succinic acid, dimer and trimer acids, terephthalic acid, phthalic acid, and mixtures of these diols and acids.

105-118. (canceled)
